(19) United States
(12) Patent Application Publication
(10) Pub. No.: US 2008/0193700 A1
(43) Pub. Date: Aug. 14, 2008

(54) Metal Chelates and Their Use in Optical Recording Media Having High Storage Capacity

(75) Inventors: Heinz Wolleb, Fehren (CH); Annemarie Wolleb, Fehren (CH); Frank Bienewald, Hegenheim (FR); Beat Schmidhalter, Bubendorf (CH); Jean-Luc Budry, Rossemaison (CH)

Correspondence Address: JoAnn Villamizar;Ciba Corporation/Patent Department, 540 White Plains Road, P.O. Box 2005, Tarrytown, NY, 10591, US

(73) Assignee: CIBA SPECIALTY CHEMICALS HOLDING INC., Basel (CH)

(21) Appl. No.: 11/579,261

(22) PCT Filed: Apr. 25, 2005
(86) PCT No.: PCT/EP05/51825

§ 371 Date: Nov. 1, 2006

(30) Foreign Application Priority Data
May 5, 2004 (EP) 04101933.2

(51) Int. Cl. B32B 3/00; C07D 277/64; C07D 401/06; C07F 9/6541

(52) U.S. Cl. 428/64.4; 548/180; 546/270.1; 548/113

(57) ABSTRACT

The invention relates to novel optical recording media, which comprise particular novel metal chelates and have an excellent recording and playback quality, especially at a wavelength of from 300 to 500 nm.

The invention relates to novel optical recording media that comprise specific novel metal chelates and have excellent recording and playback quality, especially at a wavelength of from 300 to 500 nm. Recording and playback can very advantageously take place at the same wavelength, and the storage density achievable is appreciably higher than has been customary hitherto. In addition, the media according to the invention have very good storage properties before and after recording, even under especially harsh conditions, such as exposure to sunlight or fluorescent lighting, to heat and/or to high humidity. They can also be produced simply and with good reproducibility using customary coating methods, such as spin-coating.

JP 11/034 500 A discloses optical recording materials comprising metal complex dyes in combination, for example, with phthalocyanines, which can be used at from 520 to 690 nm (i.e. CD-R or DVD-R), the metal complex dyes disclosed including heterocyclic compounds such as, for example,

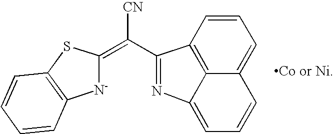

The optical properties, especially the spectral properties in or near the UV range necessary for the highest possible storage density, are not, however, able to meet the exacting requirements to a satisfactory extent. The information density per unit area is accordingly far lower than is desirable when a laser of a wavelength of from 300 to 500 nm is used.

US 2004/0 029 040 discloses optical recording media that can be used at a wavelength of about 405 nm. There are used, as the recording layer, compounds of the general formula

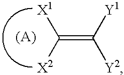

such as, for example,

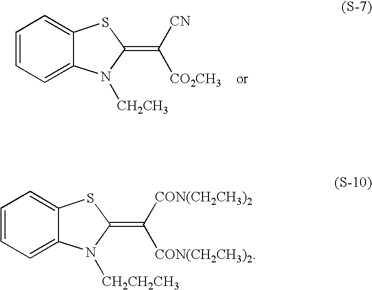

Those compounds may, if desired, be substituted by dissociable groups that optionally have, as counterion, alkali metal or metal complex ions, such as bisbenzene-1,2-dithiolatonickel(III). Those media are written at a speed of only 3.5 m·s⁻¹.

Although those compounds are ostensibly used also in pure form, it is usually intended that a ¹O₂-quencher will be added, especially in amounts of from 5 to 25% by weight, in order to improve the light stability (paragraphs [0065] and [0070]). This leads to a deterioration in the recording quality, however, when a laser of a wavelength of from 300 to 500 nm is used. In view of the crystallisation tendency generally being too high, it is in addition not possible for, for example, S-7, to be applied by spin-coating to produce a usable recording layer. Furthermore, there is insufficient disclosure of the preparation of those compounds: in spite of several experiments analogous to customary methods known to the person skilled in the art, it has so far not been possible to produce, for example, S-9 and S-10 at all.

WO 01/75 873 proposes countless other light-absorbing compounds for use with a laser of a wavelength of from 300 to 500 nm, including, for example,

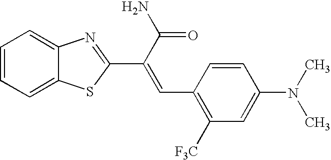

(page 54). The absorption band is, however, far too flat on the long-wavelength side, combined with an unsatisfactorily low ε-value. In addition, there are no practical examples given.

U.S. Pat. No. 6,225,023 discloses optical recording media comprising N-coordinated metal chelates of heterocyclic azo compounds, for example

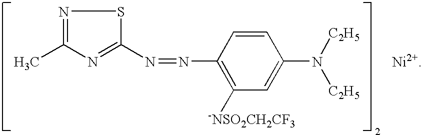

These compounds are suitable, however, only for systems where recording and playback take place using laser radiation of a wavelength far above 500 nm, for example 635 nm.

Conventional optical recording materials therefore meet only some of the exacting requirements, or do not meet all of the requirements simultaneously to complete satisfaction.

The aim of the invention is an optical recording medium with high information density and high data reliability. Such a recording medium should be robust, durable and simple to use. In addition it should be inexpensive to produce on a large scale, should require equipment that is as small and as inexpensive as possible, and should enable data to be recorded accurately and as rapidly as possible, the recording being of a quality giving reliable readability over a long period.

The invention accordingly relates to an optical recording medium comprising a substrate, a recording layer and, optionally, a reflecting layer, wherein the recording layer comprises a compound of formula Mⁿ⁺(L₁)(L₂)_{y}(L₃)_{z} (I) wherein

M is a transition metal of Groups 6 to 12 or an element of Group 13 that may additionally be coordinated with one or more further ligands and/or may optionally have an electrostatic interaction with one or more further ions inside or outside the coordination sphere in order to balance an excess charge;

n is a number 1, 2 or 3; y is the number 0 when n is 1, or is a number 0 or 1 when n is 2 or 3; z is the number 0 when n is 1 or 2, or is a number 0 or 1 when n is 3;

L₁ and L₂ are each independently of the other a ligand of formula

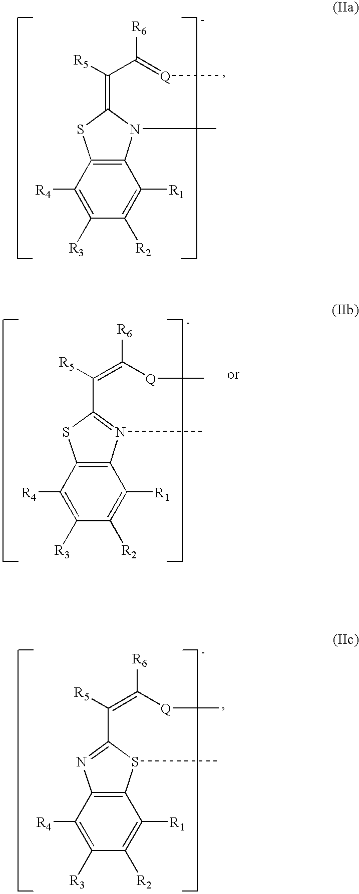

it being possible for L₁ and L₂ to be bonded to one another by any R₁, R₂, R₃, R₄, R₅, R₆ or Q;

L₃, independently of L₁ and L₂, is a further ligand (IIa), (IIb) or (IIc);

Q is O, S, NR₇, N—OR₈ or N—NR₈R₉;

R₁, R₂, R₃ and R₄ are each independently of the others R₁₀, NR₈R₉, NR₁₁NR₈R₉, NO₂, SiR₈R₁₂R₁₃, C(R₁₁)═NR₈, C(R₁₁)═N—OR₈, CON(R₁₁)OR₈, CON(R₁₁)NR₈R₉, S(O)R₁₂, S(O)₂—R₁₂, S(O)—OR₈, S(O)N(R₁₁)NR₈R₉, SO₂NR₈R₉, SO₂N(R₁₁)NR₈R₉, SO₃R₈, P(O)R₁₂R₁₃, P(O)R₁₂OR₈, P(O)OR₈OR₉ or P(O)(NR₈R₉)₂; it being possible for one of R₂, R₃ and R₄ in addition to be C₆-C₁₀aryl, C₁-C₉heteroaryl, C₇-C₁₂aralkyl or C₂-C₁₂heteroaralkyl each unsubstituted or substituted by one or more optionally identical or different, nitro, R₁₀ and/or R₇ radicals;

R₅ and R₁₀, independently of R₁ to R₄, and, where applicable, each R₁₀ independently of any other R₁₀, are hydrogen, halogen, OR₇, SR₇, NR₇R₈, COR₁₁, COOR₁₁, CONR₈R₉, CN, OCN or SCN, or C₁-C₅alkyl, C₂-C₅alkenyl, C₂-C₅alkynyl, C₃-C₅cycloalkyl, hetero-C₂-C₅cycloalkyl or C₃-C₅cycloalkenyl each unsubstituted or mono- or poly-substituted by halogen and/or by OR₁₁;

R₆, independently of R₁ to R₅, is hydrogen, OR₈, SR₈, NR₈R₉; C₁-C₅alkyl, C₂-C₅alkenyl, C₂-C₅alkynyl, C₃-C₅cycloalkyl, hetero-C₂-C₅cycloalkyl or C₃-C₅cycloalkenyl each unsubstituted or mono- or poly-substituted by COR₁₁, COOR₁₁, CONR₈R₉, CN, halogen and/or by OR₁₁; or C₆-C₁₀aryl, C₁-C₉heteroaryl, C₇-C₁₂aralkyl or C₂-C₁₂heteroaralkyl each unsubstituted or substituted by one or more optionally identical or different nitro, R₁₀ and/or R₇ radicals;

R₇ is hydrogen, COR₁₁, COOR₁₂, CR₈OR₉OR₁₁, CONR₈R₉, SO₂R₁₂, P(O)R₁₂R₁₃, P(O)R₁₂OR₁₃ or P(O)OR₁₂OR₁₃, or C₁-C₅alkyl, C₂-C₅alkenyl, C₂-C₅alkynyl, C₃-C₅cycloalkyl, hetero-C₂-C₅cycloalkyl or C₃-C₅cycloalkenyl each unsubstituted or mono- or poly-substituted by halogen and/or by OR₁₁;

or R₁ and R₂, R₂ and R₃ and/or R₃ and R₄, in each case together as a pair, are C₂-C₁₀alkylene or C₂-C₁₀alkenylene each unsubstituted or mono- or poly-substituted by halogen and/or by OR₁₁, additional rings being formed that are preferably not fully conjugated, or are

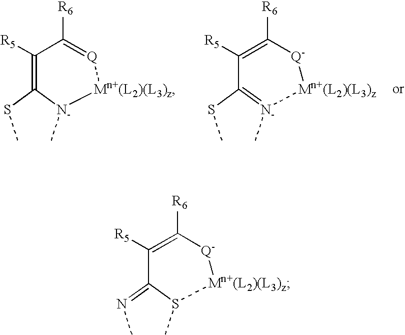

or R₅ and R₆ and/or R₆ and R₇, in each case together as a pair, are C₂-C₁₀alkylene or C₂-C₁₀alkenylene each unsubstituted or mono- or poly-substituted by halogen and/or by OR₁₁, additional rings, but not fully conjugated rings, being formed;

R₈, R₉ and R₁₁, each independently of the others and of R₁ to R₇, are hydrogen; C₁-C₅alkyl, C₂-C₅alkenyl, C₂-C₅alkynyl, C₃-C₅cycloalkyl, hetero-C₂-C₅cycloalkyl or C₃-C₅cycloalkenyl each unsubstituted or mono- or poly-substituted by halogen and/or by OR₁₁; or C₆-C₁₀aryl, C₁-C₉heteroaryl, C₇-C₁₂aralkyl or C₂-C₁₂heteroaralkyl each unsubstituted or substituted by one or more optionally identical or different halogen, OR₁₂, SR₁₂, NR₁₂R₁₃, CN, OCN, SCN, COR₁₂, CR₁₄OR₁₂OR₁₃, COOR₁₂, CONR₁₂R₁₃, SO₂R₁₂, P(O)R₁₂R₁₃, P(O)R₁₂OR₁₃ and/or P(O)OR₁₂OR₁₃ radicals;

or R₇ and R₈ and/or R₈ and R₉ together are C₂-C₁₀alkylene or C₂-C₁₀alkenylene, each unsubstituted or mono- or poly-substituted by halogen and/or by OR₁₁ and each of which may be interrupted by O or by NR₁₁; and

R₁₂, R₁₃ and R₁₄, each independently of the others, are C₁-C₅alkyl, C₂-C₅alkenyl, C₂-C₅alkynyl, C₃-C₅cycloalkyl, hetero-C₂-C₅cycloalkyl or C₃-C₅cycloalkenyl each unsubstituted or mono- or poly-substituted by halogen and/or by OR₁₁, or R₁₂ and R₁₃ together are C₂-C₁₀alkylene or C₂-C₁₀alkenylene each unsubstituted or mono- or poly-substituted by halogen and/or by OR₁₁ and each of which may be interrupted by O or by NR₁₁.

Halogen is chlorine, bromine, fluorine or iodine, preferably fluorine, chlorine or bromine, especially fluorine (for example in trifluoromethyl, α,α-difluoroethyl, β,β,β-trifluoroethyl or perfluorinated alkyl groups, such as heptafluoropropyl).

Alkyl, cycloalkyl, alkenyl or cycloalkenyl may be straight-chain or branched, monocyclic or polycyclic. Alkyl is, for example, methyl, straight-chain C₂-C₅alkyl or preferably branched C₃-C₅alkyl. Alkenyl is, for example, straight-chain C₂-C₅alkenyl or branched C₃-C₅alkenyl. C₁-C₅alkyl is accordingly, for example, methyl, ethyl, n-propyl, isopropyl, n-butyl, sec-butyl, isobutyl, tert-butyl, n-pentyl, 2-pentyl, 3-pentyl or 2,2-dimethylpropyl. C₃-C₅Cycloalkyl is, for example, cyclopropyl, cyclobutyl or cyclopentyl.

Heterocycloalkyl is cycloalkyl in which one or more carbon atom(s), but not all carbon atoms, has/have been replaced by elements of groups 13 to 16, for example by nitrogen, oxygen or sulfur atoms. Oxa- and thia-cycloalkyl, such as, for example, epoxides, episulfides, oxetyl, thietyl and tetrahydrofuryl, or N-alkylated aziridines, such as 2-(1-aza-1-ethyl)-cyclopropyl or 2-(1-aza-1-methyl)-cyclopropylmethyl, are preferred.

C₂-C₅Alkenyl and C₃-C₅cycloalkenyl are C₂-C₅alkyl and C₃-C₅cycloalkyl respectively, each of which is mono- or di-unsaturated, it being possible for two double bonds, where present, to be isolated or conjugated, for example vinyl, allyl, 2-propen-2-yl, 2-buten-1-yl, 3-buten-1-yl, 1,3-butadien-2-yl, 2-cyclobuten-1-yl, 2-penten-1-yl, 3-penten-2-yl, 2-methyl-1-buten-3-yl, 2-methyl-3-buten-2-yl, 3-methyl-2-buten-1-yl, 1,4-pentadien-3-yl or 2-cyclopenten-1-yl

Alkylene may likewise be straight-chain or branched and is, for example, methylene, straight-chain C₂-C₁₀alkylene or preferably C₃-C₁₀alkylene, which may, where applicable, be mono- or poly-branched. C₁-C₁₀Alkylene is accordingly, for example, methylene, methylidene, ethylene, 1,2-propylene, 1,3-propylene, 2-methyl-1,2-propylene, 2-methyl-1,3-propylene, 3-methyl-1,3-propylene, 1,2-butylene, 1,3-butylene, 2,3-butylene, 1,4-butylene or any desired isomer of pentylene, hexylene, heptylene, octylene, nonylene or decylene, such as the various stereoisomers of 2,3,4,5-tetramethyl-2,5-hexylene.

C₂-C₁₀Alkenylene is C₂-C₁₀alkylene that is mono- or di-unsaturated, wherein two double bonds may, where present, be isolated or conjugated, with the proviso, however, that full conjugation does not result. Where applicable, the conjugation of the π-system must be interrupted by at least one fully saturated carbon atom in the bridge between the two free valencies of the alkenylene di-radical. That fully saturated carbon atom may itself have a free valency, or may be located elsewhere in the bridge between those two carbon atoms having free valencies. C₂-C₆Alkenylene is accordingly, for example, 1-propen-1,3-ylene, 1-buten-1,3-ylene, 1-buten-1,4-ylene, 2-buten-1,4-ylene, 3-buten-1,3-ylene, 1-penten-1,3-ylene, 1-penten-1,4-ylene, 1-penten-1,5-ylene, 1-penten-2,3-ylene, 1-penten-2,4-ylene, 1-penten-2,5-ylene, 1-penten-3,4-ylene, 1-penten-3,5-ylene, 1-penten-4,5-ylene, 2-penten-1,3-ylene, 2-penten-1,4-ylene, 2-penten-1,5-ylene, 2-penten-2,4-ylene, 2-penten-2,5-ylene, 2-penten-3,4-ylene, 2-penten-3,5-ylene, 2-penten-4,5-ylene, 1,3-pentadien-1,5-ylene, 1,3-pentadien-2,5-ylene, 1,3-pentadien-3,5-ylene, 1,3-pentadien-4,5-ylene, 1,4-pentadien-1,3-ylene, 1,4-pentadien-1,4-ylene, 1,4-pentadien-1,5-ylene, 1,4-pentadien-2,4-ylene, 1-hexen-1,3-ylene, 1-hexen-1,4-ylene, 1-hexen-1,5-ylene, 1-hexen-1,6-ylene, 1-hexen-2,3-ylene, 1-hexen-2,4-ylene, 1-hexen-2,5-ylene, 1-hexen-2,6-ylene, 1-hexen-3,4-ylene, 1-hexen-3,5-ylene, 1-hexen-3,6-ylene, 1-hexen-4,5-ylene, 1-hexen-4,6-ylene, 1-hexen-5,6-ylene, 2-hexen-1,3-ylene, 2-hexen-1,4-ylene, 2-hexen-1,5-ylene, 2-hexen-1,6-ylene, 2-hexen-2,4-ylene, 2-hexen-2,5-ylene, 2-hexen-2,6-ylene, 2-hexen-3,4-ylene, 2-hexen-3,5-ylene, 2-hexen-3,6-ylene, 2-hexen-4,5-ylene, 2-hexen-4,6-ylene, 2-hexen-5,6-ylene, 3-hexen-1,2-ylene, 3-hexen-1,3-ylene, 3-hexen-1,4-ylene, 3-hexen-1,5-ylene, 3-hexen-1,6-ylene, 3-hexen-2,3-ylene, 3-hexen-2,4-ylene, 3-hexen-2,5-ylene, 3-hexen-2,6-ylene, 3-hexen-3,5-ylene, 3-hexen-3,6-ylene, 3-hexen-4,5-ylene, 3-hexen-4,6-ylene, 3-hexen-5,6-ylene, 1,3-hexadien-1,5-ylene, 1,3-hexadien-1,6-ylene, 1,3-hexadien-2,5-ylene, 1,3-hexadien-2,6-ylene, 1,3-hexadien-3,5-ylene, 1,3-hexadien-3,6-ylene, 1,3-hexadien-4,5-ylene, 1,3-hexadien-4,6-ylene, 1,3-hexadien-5,6-ylene, 1,4-hexadien-1,3-ylene, 1,4-hexadien-1,4-ylene, 1,4-hexadien-1,5-ylene, 1,4-hexadien-1,6-ylene, 1,4-hexadien-2,3-ylene, 1,4-hexadien-2,4-ylene, 1,4-hexadien-2,5-ylene, 1,4-hexadien-2,6-ylene, 1,4-hexadien-3,4-ylene, 1,4-hexadien-3,5-ylene, 1,4-hexadien-3,6-ylene, 1,4-hexadien-4,6-ylene, 1,4-hexadien-5,6-ylene, 1,5-hexadien-1,3-ylene, 1,5-hexadien-1,4-ylene, 1,5-hexadien-1,5-ylene, 1,5-hexadien-1,6-ylene, 1,5-hexadien-2,3-ylene, 1,5-hexadien-2,4-ylene, 1,5-hexadien-2,5-ylene, 1,5-hexadien-3,4-ylene, 2,4-hexadien-1,2-ylene, 2,4-hexadien-1,3-ylene, 2,4-hexadien-1,4-ylene, 2,4-hexadien-1,5-ylene or 2,4-hexadien-1,6-ylene, but is not, for example, 1,2-ethylene, butadien-1,3-ylene or butadien-1,4-ylene. The same principle applies analogously also to higher analogues thereof and to C₇-C₁₀alkenylene.

C₇-C₁₂Aralkyl is, for example, benzyl, 2-benzyl-2-propyl, β-phenyl-ethyl, α,α-dimethylbenzyl, ω-phenyl-butyl or ω-phenyl-hexyl, preferably benzyl. When C₇-C₁₂aralkyl is substituted, substitution may be on the alkyl or the aryl moiety of the aralkyl group, the latter alternative being preferred.

C₆-C₁₀Aryl is, for example, phenyl, naphthyl or biphenylyl, preferably phenyl.

C₂-C₉Heteroaryl is an unsaturated or aromatic radical having 4n+2 conjugated π-electrons, for example 2-thienyl, 2-furyl, 2-pyridyl, 2-thiazolyl, 2-oxazolyl, 2-imidazolyl, isothiazolyl, thiadiazolyl, triazolyl, tetrazolyl or any other ring system consisting of thiophene, furan, pyridine, thiazole, thiadiazole, oxazole, imidazole, isothiazole, triazole, pyridine and benzene rings that is unsubstituted or substituted by from 1 to 6 ethyl, methyl, ethylene and/or methylene radicals, for example benzotriazolyl, and, in the case of N-heterocycles, optionally also in the form of N-oxides thereof.

C₂-C₁₂Heteroaralkyl is, for example, C₁-C₈alkyl substituted by C₁-C₁₁heteroaryl.

In addition, aryl and aralkyl may also be aromatic groups bonded to a metal, for example in the form of transition metal metallocenes, known per se, more especially

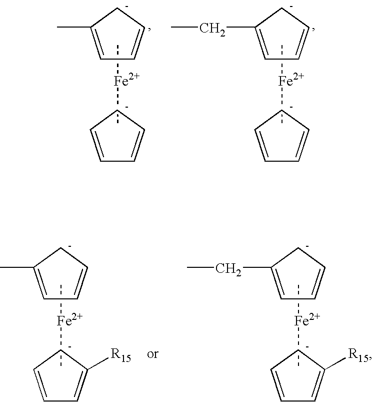

wherein R₁₅ is CH₂OH, CH₂OR₁₄ or COOR₁₄.

The compound of formula (I) may also be a dimer or oligomer, two or more radicals of formula (I) being bonded to one another by direct bonds between substituents or by bridges with C₂-C₁₀alkylene or C₂-C₁₀alkenylene in accordance with the definitions given hereinbefore. In that case too, of course, advantageously full conjugation should not occur. Oligomers preferably consist of 3, 4 or 5 radicals of formula (I) and may be cyclic or may comprise, as terminal groups, non-metallated radicals of formula (I) that are coordinated with only one radical of formula (I) and other ligands and/or are not bridged.

M is advantageously a transition metal of Groups 6 to 12, preferably a transition metal of Groups 8 to 12, especially a transition metal of Groups 9 to 11 (new IUPAC nomenclature), for example Au, Cd, Co, Cu, Cr, Ir, Mn, Mo, Ni, Fe, Os, Pd, Pt, Re, Rh, Ru, W or Zn, especially Co, Cu or Ni, more especially Co(II), Cu(II) or Ni(II). More especially preferred is Co, especially Co(II). In compounds of formula (I) wherein z is 1, the transition metal cation is preferably a relatively large cation, such as Ir³⁺ or R³⁺.

Depending on the number of electrons in the outermost d-shell, such transition metals may be coordinated with further ligands. The further ligands are, for example, known compounds, for example ammonia, acetylacetone, water, amines, polyamines, alcohols, polyalcohols or olefins.

The compounds of formula (I) are advantageously electron-neutral, which on no account excludes the presence of cations and anions, provided that their charges balance one another. They may be either pairs of ions or, alternatively, zwitterions.

Preferably, at least two of R₁, R₂, R₃ and R₄ are hydrogen;

R₅ is preferably hydrogen;

R₆ is preferably C₁-C₅alkyl, C₂-C₅alkenyl, C₂-C₅alkynyl, C₃-C₅cycloalkyl, hetero-C₂-C₅cycloalkyl or C₃-C₅cycloalkenyl mono- or poly-substituted by halogen and/or by OR₁₁, or phenyl;

R₇ is preferably COR₁₁, COOR₁₂, SO₂R₁₂, P(O)R₁₂R₁₃, P(O)R₁₂OR₁₃ or P(O)OR₁₂OR₁₃;

R₈ and R₉ are preferably each independently of the other hydrogen or alkyl, alkenyl, alkynyl, cycloalkyl, heterocycloalkyl or cycloalkenyl;

R₁₁ is preferably hydrogen or C₁-C₃alkyl, especially hydrogen, methyl or ethyl;

R₁₂ and R₁₃ are preferably each independently of the other C₁-C₅alkyl or C₂-C₅alkenyl;

n is preferably the number 2 and y is preferably the number 1;

L₁ and L₂ are preferably ligands of formula (IIa) or (IIb); and/or

Q is preferably O or NR₇, especially O.

Especially preferably, R₁ is hydrogen or fluorine, more especially H, and/or at least three of R₁, R₂, R₃ and R₄ are hydrogen.

Alkyl, alkenyl, alkynyl, cycloalkyl, heterocycloalkyl or cycloalkenyl at any position is preferably methyl, ethyl, n-propyl, isopropyl, vinyl, allyl, propargyl, cyclopropyl, 2-oxacyclopropyl or 2-thiacyclopropyl, especially trifluoromethyl, α,α,-difluoroethyl, β,β,β-trifluoroethyl or heptafluoropropyl, especially preferably as a meaning of R₆.

Those preferred meanings apply both individually and in any desired combination. Generally, the more of the preferred individual features any compound according to the invention has, the more advantageous the properties it exhibits.

The substantial versatility of the invention is evident from the following purely illustrative examples:

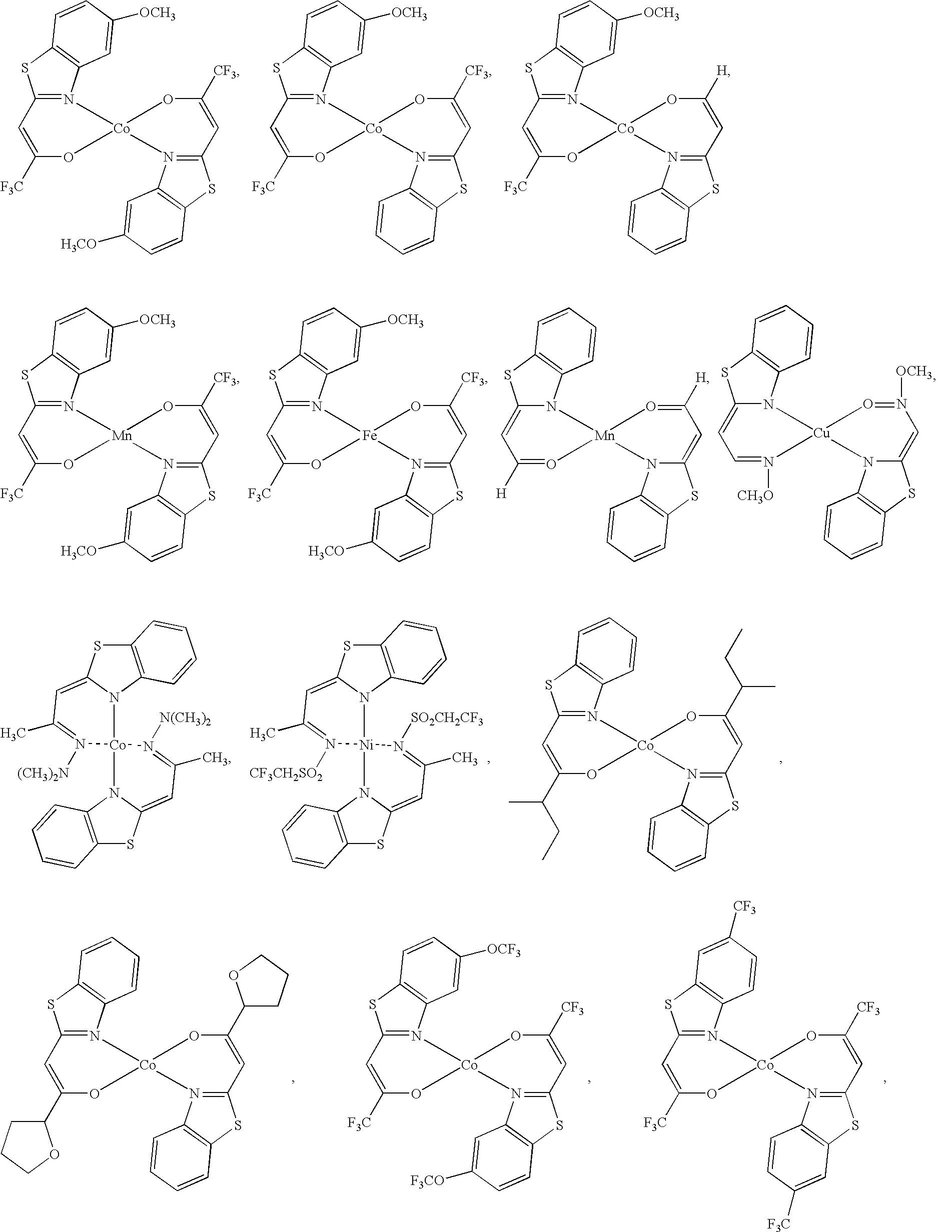

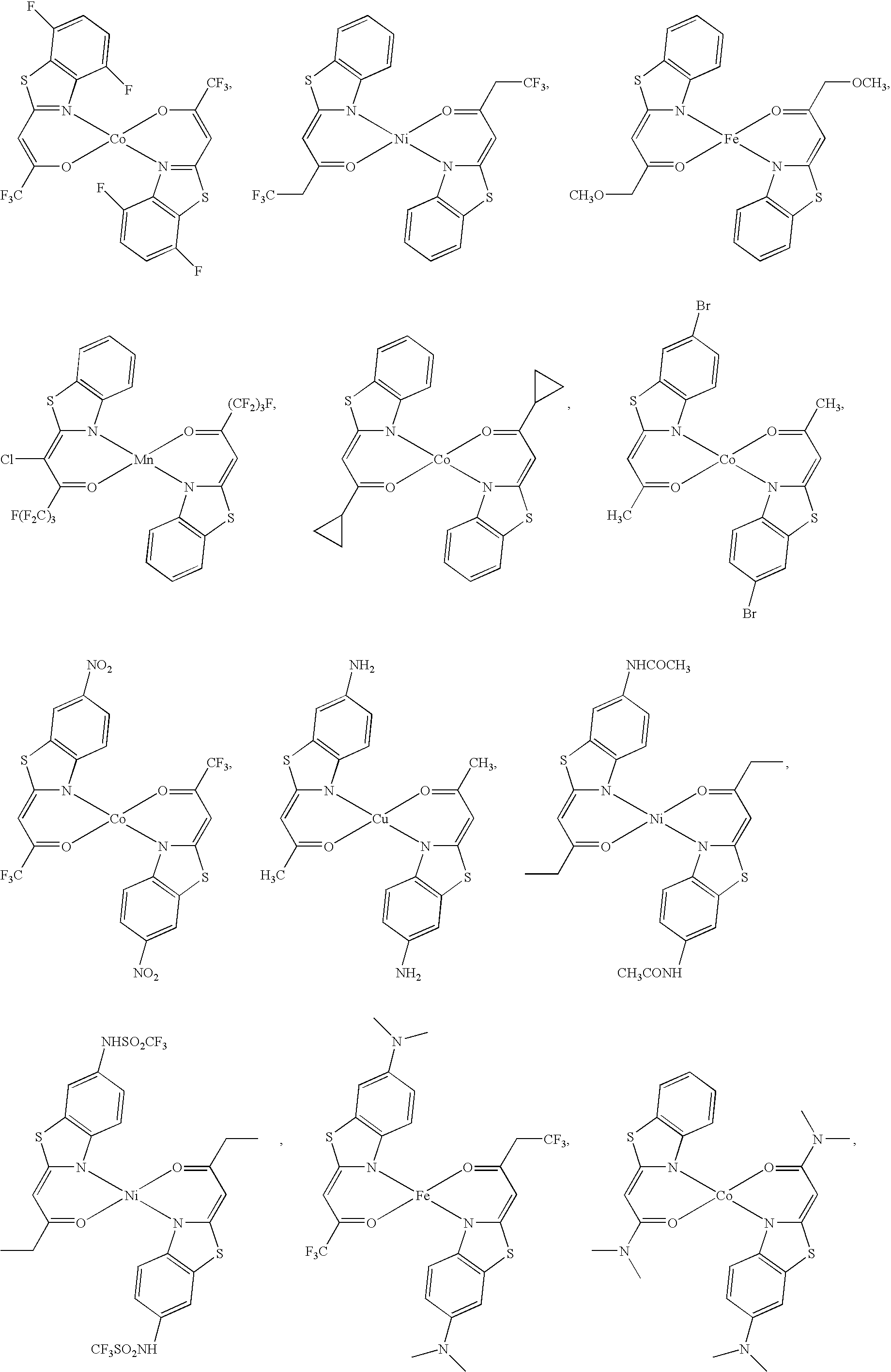

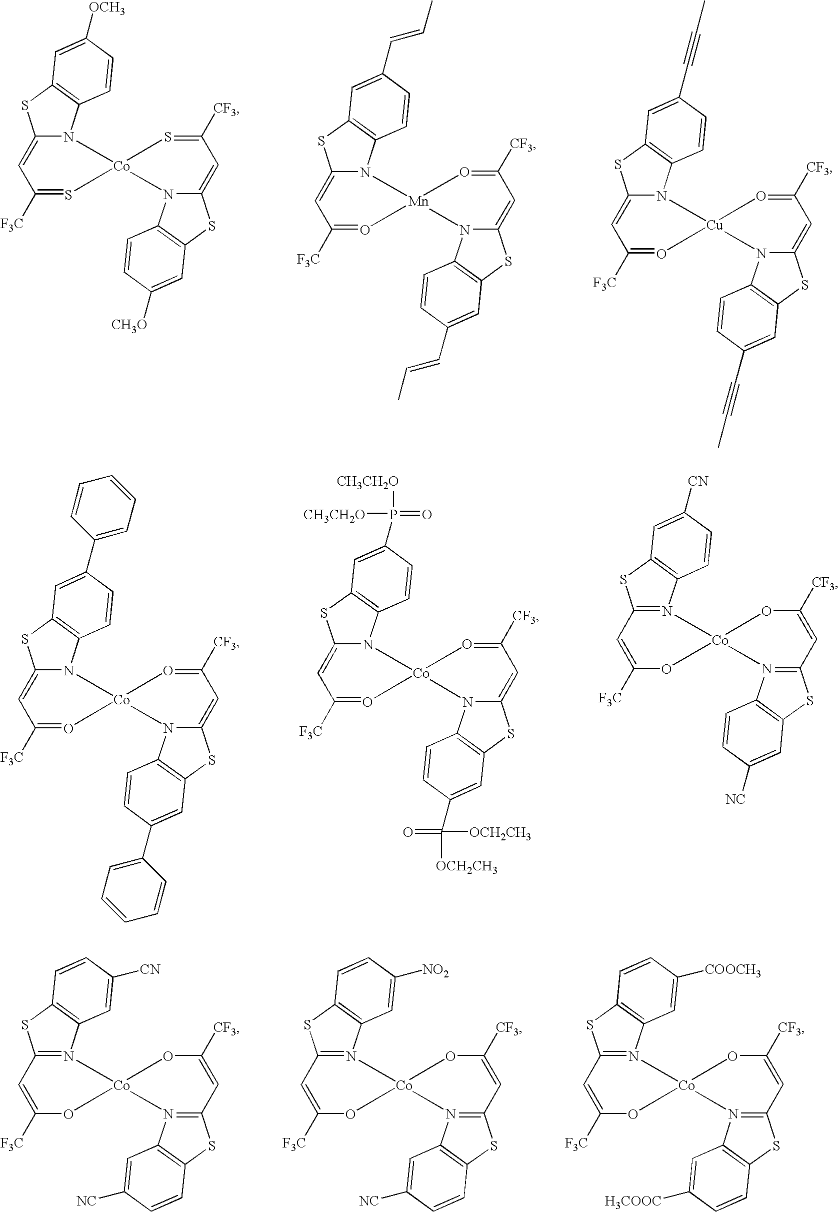

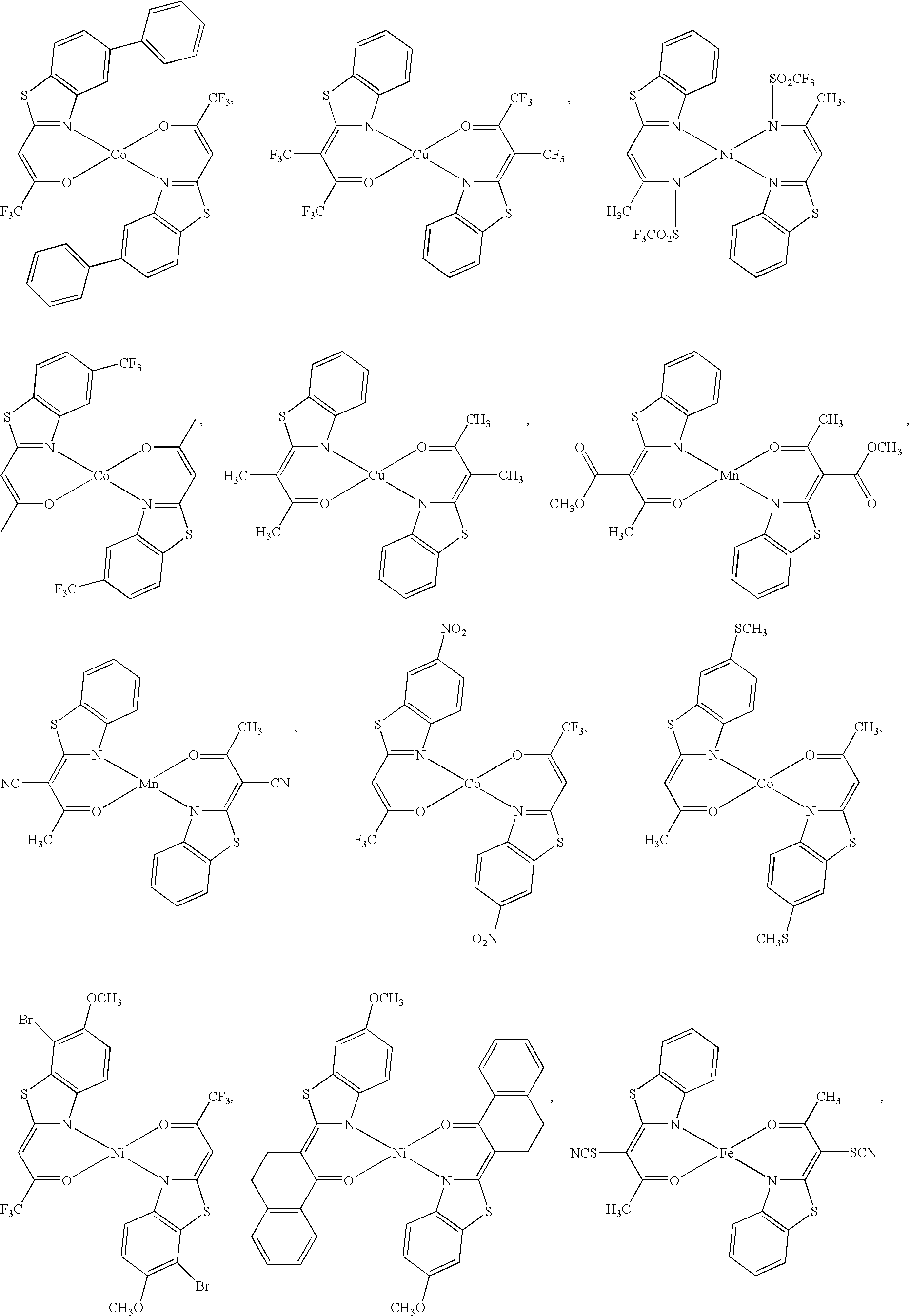

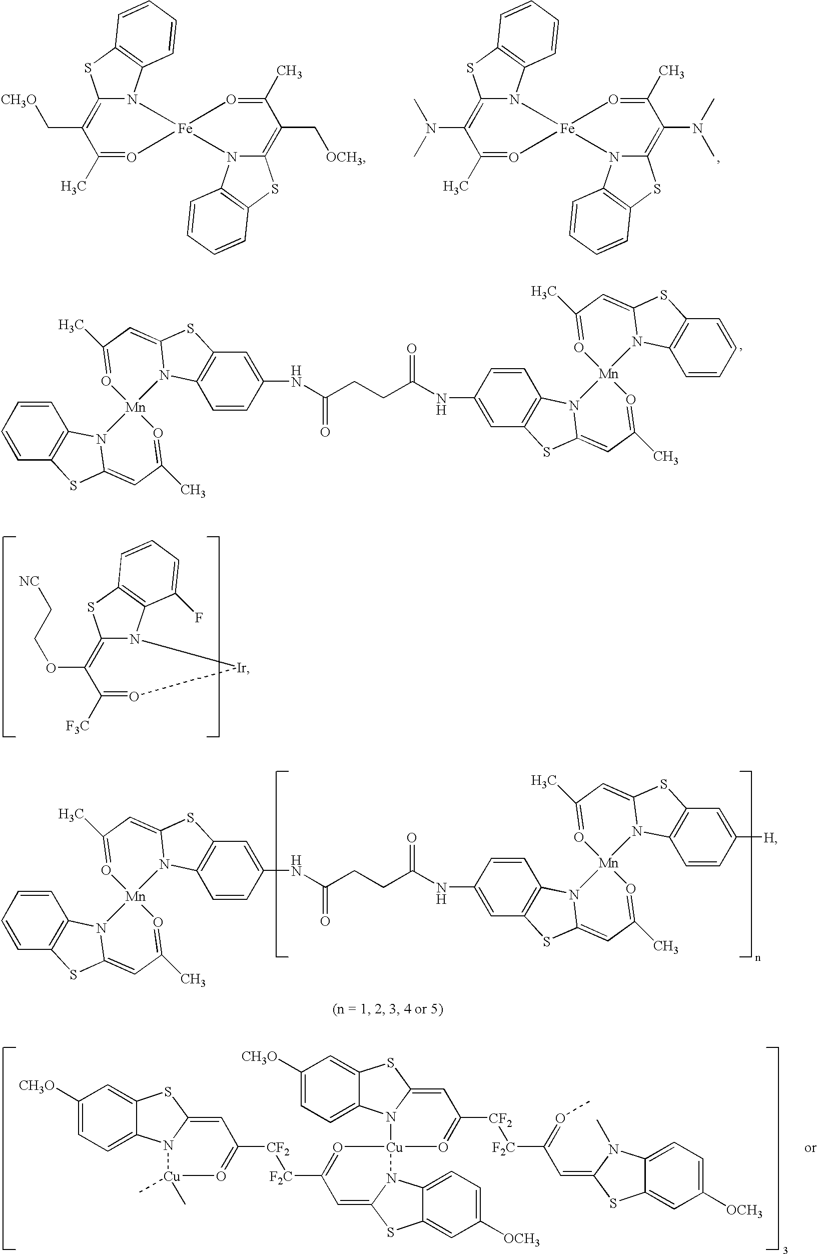

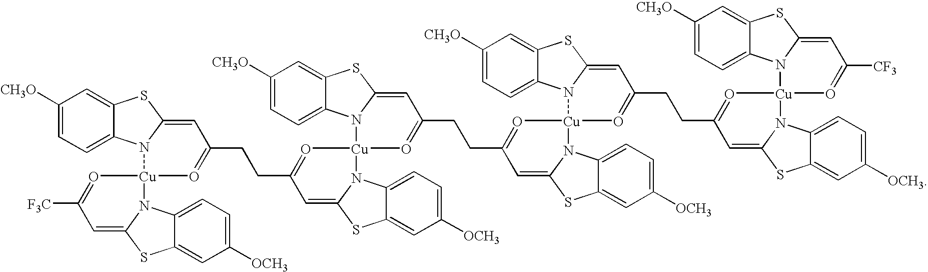

Further compounds are mentioned hereinbelow in the form of detailed examples. It will be understood that there may be present on the metal also further ligands, which are usually not very strongly bonded and therefore are reversibly removable.

The recording layer advantageously comprises a compound of formula (I) or a mixture of such compounds as the main constituent or at least as a significant component, for example from 1 to 100% by weight, preferably from 20 to 100% by weight, especially from 50 to 100% by weight. Further customary constituents are possible, such as, for example, other chromophores (for example those having an absorption maximum at from 300 to 1000 nm), stabilisers, ¹O₂-, triplet- or luminescence-quenchers, melting point reducers, decomposition accelerators or any other additives that have already been described in optical recording media. Preferably, stabilisers or fluorescence quenchers are optionally added.

Chromophores that can optionally be used in the recording layer in addition to the compounds of formula (I) are, for example, cyanines and cyanine metal complexes (U.S. Pat. No. 5,958,650), aza- and phospha-cyanines (WO 02/082438), styryl compounds (U.S. Pat. No. 6,103,331), oxonol dyes (EP 0 833 314), azo dyes and azo metal complexes (JP 11/028,865 A), phthalocyanines (EP 0 232 427, EP 0 337 209, EP 0 373 643, EP 0 463 550, EP 0 492 508, EP 0 509 423, EP 0 511 590, EP 0 513 370, EP 0 514 799, EP 0 518 213, EP 0 519 419, EP 0 519 423, EP 0 575 816, EP 0 600 427, EP 0 676 751, EP 0 712 904, WO 98/14 520, WO 00/09 522, WO 02/25 648, WO 02/083 796, EP 1 253 586, EP 1 265 233, EP 1 271 500, WO 05/000 972), porphyrins, porphyrazines (EP 0 822 546, U.S. Pat. No. 5,998,093, JP 2001/277 723 A, WO 03/042 990), carbopyronines (WO 03/007 296), dipyrromethene dyes and metal chelate compounds thereof (EP 0 822 544, EP 0 903 733), xanthene dyes and metal complex salts thereof (U.S. Pat. No. 5,851,621, WO 03/098 617, WO 03/098 618), pyridone metal complexes (WO 03/063 151) or squaric acid compounds (EP 0 568 877), and also oxazines, dioxazines, diazastyryls, formazanes, anthraquinones or phenothiazines; this list is on no account exhaustive and the person skilled in the art will interpret the list as including further known dyes, for example those from WO 04/088 649 or PCT/EP 05/050 673.

Further, preferred chromophores that can optionally be used in the recording layer in addition to the compounds of formula (I) are known UV absorbers, such as, for example, azacyanines (JP H11/34 500), merocyanines (WO 02/080 161), triazines (JP 2001/277 720, JP 2002/160 452, WO 04/106 311, JP 2004/160 883), salicylaldehydes (JP 2004/034 645), stilbenes (JP 2003/246 142), other substituted olefins (US 2004/0 290 401) or metal chelates (WO 04/079 732, WO 05/012 228).

Mixtures are known to have a number of advantages, for example better solubility and a lower tendency towards crystallisation, so that it is easier to produce stably amorphous layers by spin-coating. By optimisation of the mixing ratios in a manner known per se there are obtained solid recording layers having advantageous thermal and optical properties, especially having steep absorption bands. In addition, it is therefore often possible to counteract the flattening of the spectral absorption edge in the solid state. Optimum mixing ratios are accordingly generally determined by series tests, in which different groove geometries are also included.

It will be understood that, where applicable, preference is given to those additional dyes which are themselves known for use in optical recording materials at from 300 to 500 nm. Special preference is given to mixtures of the compounds of formula (I), for example mixtures of homologues and mixtures comprising both compounds of formula (I) wherein L₁, L₂ and/or L₃ are identical and compounds of formula (I) wherein L₁, L₂ and/or L₃ are different, such as M⁺(L₁)⁻, M²⁺(L₁)⁻, M³⁺(L₁)⁻, M²⁺(L₁)⁻(L₁)⁻, M²⁺(L₂)⁻(L₂)⁻, M²⁺(L₁)⁻(L₂)⁻, M³⁺(L₁)⁻(L₁, M³⁺(L₂)⁻(L₂), M³⁺(L₁)⁻(L₂)⁻, M³⁺(L₁)⁻(L₁)⁻(L₁)⁻, M³⁺(L₂)⁻(L₂)⁻(L₂)⁻, M³⁺(L₃)⁻(L₃)⁻(L₃), M³⁺(L₁)⁻(L₂)⁻(L₁)⁻, M³⁺(L₁)⁻(L₁)⁻(L₃)⁻, M³⁺(L₁)⁻(L₂)⁻(L₂)⁻, M³⁺(L₂)⁻(L₂)⁻(L₃)⁻, M³⁺(L₁)⁻(L₃)⁻(L₃)⁻, M³⁺(L₃)⁻(L₂)⁻(L₃)⁻ and M³⁺(L₁)⁻(L₂)⁻(L₃)⁻, each either with additional, neutral and/or anionic ligands or without additional ligands.

When the recording layer comprises other chromophores that are not suitable per se for use at from 300 to 500 nm, the amount of such chromophores should preferably be small, so that the absorption thereof at the wavelength of the inflection point (point of maximum gradient) of the gradient of the long-wavelength flank of the absorption band of the solid layer as a whole, which is a decisive factor for the recording, is a fraction of the absorption of the compounds of formula (I) in the solid layer as a whole at the same wavelength, advantageously at most ⅓, preferably at most ⅕, especially at most 1/10. The absorption maximum of dye mixtures in the spectral range from 300 to 500 nm is preferably at a wavelength lower than 450 nm, preferably lower than 400 nm, especially at 340-380 nm.

Stabilisers and ¹O₂-, triplet- or luminescence-quenchers are, for example, metal complexes of N- or S-containing enolates, phenolates, bisphenolates, thiolates or bisthiolates or of azo, azomethine or formazan dyes, such as bis(4-dimethylamino-dithiobenzil)nickel [CAS N^{o} 38465-55-3], ®Irgalan Bordeaux EL, ®Cibafast N or similar compounds, hindered phenols and derivatives thereof, such as ®Cibafast AO, o-hydroxyphenyl-triazoles or -triazines or other UV absorbers, such as ®Cibafast W or ®Cibafast P or hindered amines (TEMPO or HALS), also as nitroxides or NOR-HALS, also diimmonium salts, Paraquat™ salts or Orthoquat™ salts, such as ®Kayasorb IRG 022, ®Kayasorb IRG 040, or optionally also radical salts, such as N,N,N′,N′-tetrakis(4-dibutylaminophenyl)-p-phenyleneamine-ammonium salts. The latter are available from Organica (Wolfen/DE); ®Kayasorb brands are available from Nippon Kayaku Co. Ltd., and ®Irgalan and ®Cibafast brands are available from Ciba Spezialitätenchemie AG.

Many such structures are known, some of them also in connection with optical recording media, for example from U.S. Pat. No. 5,219,707, JP 06/199045 A, JP 07/76169 A, JP 07/262,604 A or JP 2000/272241 A. They may be, for example, salts of metal complex anions, as disclosed in the above-mentioned publications, or metal complexes, illustrated, for example, by the compound of formula

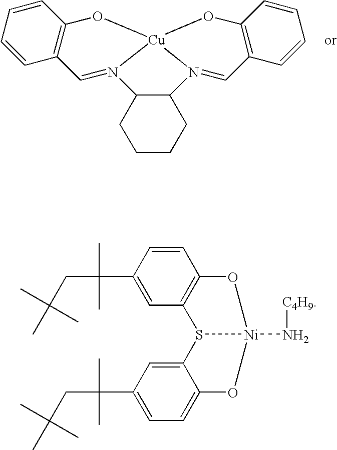

The person skilled in the art will know from other optical information media or, based on his general knowledge and the prior art, will easily identify, which additives in which concentration are particularly well suited to which purpose. Suitable concentrations of additives are, for example, from 0.001 to 1000% by weight, preferably from 1 to 50% by weight, based on the recording medium of formula (I).

The optical recording materials according to the invention exhibit, overall, excellent spectral properties of the solid amorphous recording layer, and also the refractive index is surprisingly high. By virtue of an aggregation tendency in the solid that is surprisingly low for such compounds, the absorption band is narrow and intense, the absorption band being especially steep on the long-wavelength side. Crystallites are unexpectedly and advantageously not formed or are formed only to a negligible extent. The reflectivity of the layers in the range of the writing and reading wavelength is high in the unwritten state. The sensitivity to laser radiation is high in the writing mode; the stability with respect thereto in the lower-energy readout mode is high.

By virtue of those excellent layer properties it is possible to obtain a rapid optical recording having high sensitivity, high reproducibility and geometrically very precise mark boundaries, the refractive index and the reflectivity changing substantially, which gives a high degree of contrast. The differences in the mark lengths and the interval distances (“jitter”) are surprisingly small both at normal recording speed (about from 4.5 to 5.5 m·s⁻¹) and at a higher recording speed (for example from 9 m·s⁻¹ to 25 m·s⁻¹ or even higher), which enables a high storage density to be obtained using a narrow recording channel with a relatively small track spacing (“pitch”). In addition, the recorded data are played back with an astonishingly low error rate, so that relatively short marks are possible, including, for example, those of length 0.15±0.01 μm (2T) in conformity with the Blu-Ray™ Standard, and error correction requires only a small amount of storage space.

By virtue of the excellent solubility, including in some cases also in non-polar solvents, solutions can be used even in high concentrations without troublesome precipitation, for example during storage, so that problems during spin-coating are largely eliminated. This applies especially to compounds containing branched C₃-C₅alkyl.

Recording and playback can take place at the same wavelength with a laser source of advantageously from 300 to 500 nm, especially from 350 to 500 nm, preferably from 370 to 450 nm. Especially preferred is the UV range from 370 to 390 nm, especially approximately 380 nm, or especially at the edge of the visible range from 390 to 430 nm, more especially approximately 405±5 nm. In the field of compact, blue or violet laser diodes (such as Nichia GaN 405 nm) with an optical system of high numerical aperture (for example 0.85) the marks can be so small and the tracks so narrow that about 20 to 30 Gb per recording layer is achievable on a 120 mm disc. At 380 nm it is possible to use, for example, indium-doped UV-VCSELs (Vertical-Cavity Surface-Emitting Laser), (Jung Han et al., see MRS Internet J. Nitride Semicond. Res. 5S1, W6.2 [2000]).

The invention therefore relates also to a method of recording or playing back data, wherein the data on an optical recording medium according to the invention are recorded or played back at a wavelength of from 300 to 500 nm. The recording preferably takes place at a linear speed v of at least 4.5 m·s⁻¹, there especially being created marks of different lengths, the shortest of which are almost circular and the longest of which are of a length corresponding to approximately four times the width. The linear speed is especially at least 9 m·s⁻¹ (1×), 18 m·s⁻¹ (2×) or 36 m·s⁻¹ (4×).

The recording medium can be based on the structure of known recording media and in that case is, for example, analogous to those mentioned above, such as DVD+R or DVD-R. It may therefore be composed, for example, of a transparent substrate, a recording layer comprising at least one of the compounds of formula (I), a reflector layer and a covering layer, the writing and readout being effected through the substrate. Such a system suitable for recording and playback at a wavelength of from 300 to 500 nm is, for example, HD DVD™ (formerly known as advanced optical disk AOD).

Suitable substrates are, for example, glass, minerals, ceramics and thermosetting and thermoplastic plastics. Preferred supports are glass and homo- or co-polymeric plastics. Suitable plastics are, for example, thermoplastic polycarbonates, polyamides, polyesters, polyacrylates and polymethacrylates, polyurethanes, polyolefins, polyvinyl chloride, polyvinylidene fluoride, polyimides, thermosetting polyesters and epoxy resins. Special preference is given to polycarbonate substrates, which can be produced, for example, by injection-moulding. The substrate can be in pure form or may comprise customary additives, for example UV absorbers or dyes, as proposed e.g. in JP 04/167239 A as light stabilisation for the recording layer. In the latter case it may be advantageous for the dye added to the support substrate to have no or at most only low absorption in the region of the writing wavelength (emission wavelength of the laser), preferably up to a maximum of about 20% of the laser light focussed onto the recording layer.

The substrate is advantageously transparent over at least a portion of the range from 300 to 500 nm, so that it is permeable to, for example, at least 80% of the incident light of the writing or readout wavelength. The substrate is advantageously from 10 μm to 2 mm thick, preferably from 100 to 1200 μm thick, especially from 600 to 1100 μm thick, with a preferably spiral guide groove (track) on the coating side, having a groove depth of from 10 to 200 nm, preferably from 60 to 150 nm, a groove width of from 100 to 400 nm, preferably from 150 to 250 nm, and an axial spacing between two grooves of from 200 to 600 nm, preferably from 250 to 450 nm (for example having a groove depth of 80±20 nm, a groove width of 200±50 nm and an axial spacing between two turns of 370±60 nm). Grooves of different cross-sectional shape are known, for example rectangular, trapezoidal or V-shaped. Analogously to the known CD-R and DVD±R media, the guide groove may additionally undergo a small periodic or quasi-periodic lateral deflection (wobble), so that synchronisation of the speed of rotation and the absolute positioning of the readout head (pick-up) is made possible. Instead of, or in addition to, the deflection, the same function can be performed by markings between adjacent grooves (pre-pits).

The recording medium is applied, for example, by application of a solution by spin-coating, the objective being to produce a layer that is as amorphous as possible, the thickness of which layer in the groove, depending upon the geometry of the groove, is advantageously from 20 to 150 nm, preferably from 30 to 120 nm, especially from 30 to 80 nm, and adjacent thereto (land) is advantageously from 0 to 70 nm, preferably from 1 to 20 nm, especially from 2 to 10 nm. In another embodiment, achievable using the compounds of formula (I), advantageously the thickness of the recording layer in the groove may be from 30 to 80 nm and, adjacent thereto (land), from 20 to 70 nm, the difference between the layer thicknesses in the groove and on the surface being less than 20 nm, preferably less than 10 nm. As a result it is possible, compatibly with HD-DVD-Rewritable™, to write and read both in the grooves and on the surface alongside. The track pitch is in that case only about half as great, and the total storage capacity is greater.

In both embodiments, writing and readout take place from the substrate side. The laser beam is directed onto the recording layer through the substrate and has a wavelength of preferably from 300 to 500 nm, especially from 370 to 450 nm. A reflector layer may be present on the side of the recording layer opposite from the substrate.

Reflecting materials suitable for the reflector layer include especially metals, which provide good reflection of the laser radiation used for recording and playback, for example the metals of Main Groups III, IV and V and of the Sub-Groups of the Periodic Table of the Elements. Al, In, Sn, Pb, Sb, Bi, Cu, Ag, Au, Zn, Cd, Hg, Sc, Y, La, Ti, Zr, Hf, V, Nb, Ta, Cr, Mo, W, Fe, Co, Ni, Ru, Rh, Pd, Os, Ir, Pt and the lanthanide metals Ce, Pr, Nd, Pm, Sm, Eu, Gd, Tb, Dy, Ho, Er, Tm, Yb and Lu and alloys thereof are especially suitable. On account of its high reflectivity and ease of production special preference is given to a reflective layer of aluminium, silver, gold or an alloy thereof (for example a white gold alloy or silver/chromium alloy), especially aluminium on economic and ecological grounds. The reflector layer is advantageously from 5 to 200 nm thick, preferably from 10 to 100 nm thick, especially from 20 to 80 nm thick, but reflector layers of greater thickness are also possible.

Materials suitable for the covering layer include chiefly plastics, which are applied in a thin layer to the reflector layer either directly or with the aid of adhesion promoters. It is advantageous to select mechanically and thermally stable plastics having good surface properties, which can be modified further, for example written on. The plastics may be thermosetting plastics and thermoplastic plastics. Directly applied covering layers are preferably coatings that are radiation-cured (e.g. using UV radiation), which are particularly simple and economical to produce. A wide variety of radiation-curable materials are known. Examples of radiation-curable monomers and oligomers are acrylates and methacrylates of diols, triols and tetrols, polyimides of aromatic tetracarboxylic acids and aromatic diamines having C₁-C₄alkyl groups in at least two ortho-positions of the amino groups, and oligomers having dialkylmaleimidyl groups, e.g. dimethylmaleimidyl groups. For covering layers that are applied using adhesion promoters it is preferable to use the same materials as those used for the substrate layer, especially polycarbonates. The adhesion promoters used are preferably likewise radiation-curable monomers and oligomers. Instead of the covering layer applied using an adhesion promoter, there may also be used a second substrate comprising a recording and reflector layer, so that the recording medium is playable on both sides. Preference is given to a symmetrical structure, the two parts being joined together at the reflector side by an adhesion promoter directly or by way of an intermediate layer. In that case it will be understood that the substrate will be only half as thick, so that overall the disc composed of two substrates is of approximately the same thickness as a disc consisting of only one substrate.

In such a structure, the optical properties of the covering layer, or of the covering materials, are essentially unimportant per se provided that, where applicable, curing thereof e.g. by UV radiation is assured. The function of the covering layer is to ensure the mechanical strength of the recording medium as a whole and, if necessary, the mechanical strength of thin reflector layers. If the recording medium is sufficiently robust, for example when a thick reflector layer is present, it is even possible to dispense with the covering layer altogether. The thickness of the covering layer depends upon the thickness of the recording medium as a whole, which should preferably be a maximum of about 2 mm thick. The covering layer is preferably from 10 μm to 1 mm thick.

The recording media according to the invention may also have additional layers, for example interference layers or barrier layers. It is also possible to construct recording media having a plurality of (for example from two to ten) recording layers. The structure and the use of such materials are known to the person skilled in the art. Where present, interference layers are preferably arranged between the recording layer and the reflecting layer, between the recording layer and the substrate and/or especially between the recording layer and the protecting layer and consist of a dielectric material, for example, as described in EP 0 353 393, of TiO₂, Si₃N₄, ZnS or silicone resins.

The recording media according to the invention can be produced by processes known per se, it being possible for various methods of coating to be employed depending upon the materials used and their function.

Suitable coating methods are, for example, immersion, pouring, brush-coating, blade-application and spin-coating, as well as vapour-deposition methods carried out under a high vacuum. When, for example, pouring methods are used, solutions in organic solvents are generally employed. When solvents are employed, care should be taken that the supports used are insensitive to those solvents. Suitable coating methods and solvents are described, for example, in EP 0 401 791 or EP 0 485 337.

The recording layer is applied preferably by the application of a dye solution by spin-coating, solvents that have proved satisfactory being especially alcohols, for example 2-methoxyethanol, 1-methoxy-2-propanol, 2-propanol or n-butanol, hydroxyketones, for example diacetone alcohol or 3-hydroxy-3-methyl-2-butanone, hydroxy esters, for example lactic acid methyl ester or isobutyric acid methyl ester, or preferably fluorinated alcohols, for example 2,2,2-trifluoroethanol or 2,2,3,3-tetrafluoro-1-propanol, and mixtures thereof. Further suitable solvents are disclosed, for example, in EP 0 483 387.

The application of the metallic reflector layer is preferably effected by sputtering or by vapour-deposition in vacuo. Such techniques are known and are described in specialist literature (e.g. J. L. Vossen and W. Kern, “Thin Film Processes”, Academic Press, 1978). The operation can advantageously be carried out continuously and achieves good reflectivity and a high degree of adhesiveness of the metallic reflector layer.

Recording is carried out in accordance with known methods by writing pits (marks) of fixed or, usually, variable length by means of a modulated, focussed laser beam guided at a constant or variable speed over the surface of the recording layer. Readout of information is carried out according to methods known per so by registering the change in reflection using laser radiation, for example as described in “CD-Player und R-DAT Recorder” (Claus Biaesch-Wiepke, Vogel Buchverlag, Würzburg 1992). The person skilled in the art will be familiar with the requirements.

The information-containing medium according to the invention is especially an optical information material of the WORM type. It can be used, for example, analogously to CD-R (compact disc-recordable) or DVD-R (digital video disc-recordable) in computers, and also as storage material for identification and security cards or for the production of diffractive optical elements, for example holograms. Recording media of the HD-DVD™ type allow the use of a laser having a numerical aperture of a maximum of about 0.7 (usually from 0.60 to 0.65), in which case, at a recording speed of 6.61 m·s⁻¹ (or a multiple thereof), discs of 120 mm diameter will have a storage capacity of 15 GB per recording layer.

Alternatively, however, there are also more recent recording media that differ substantially from CD-R and DVD±R and in which recording and playback take place not through the substrate but through the covering layer. Accordingly the respective roles of the covering layer and the substrate, especially the geometry and the optical properties, are reversed in comparison with the structure described above. Analogous concepts for digital video recordings in conjunction with a blue GaN laser diode are known, for example, from Proceedings SPIE-Int. Soc. Opt. Eng. 1999, 3864. Also at an advanced stage of development is Blu-ray™ (formerly Blu-ray Disc “BD”) with a recording speed of 5.0±0.3 m·s⁻¹ (probably soon a multiple thereof) and a storage capacity of 25±2 GB (see system description “Blu-ray Disc Rewritable Format version 1.0”/June 2002 and also Blu-ray.com).

The compounds of formula (I) according to the invention, however, also meet the increased demands of an inverse layer structure surprisingly well. Preference is therefore given to an inverse layer structure having the layer sequence substrate, reflector layer, recording layer and covering layer. The recording layer is therefore located between the reflector layer and the covering layer. A thin covering layer approximately from 50 to 400 μm in thickness is especially advantageous (typically 100 μm at a numerical aperture of 0.85).

Recording and reflector layers in an inverse layer structure have in principle the same functions as indicated above. The substrate usually has dimensions within the ranges indicated above. The preferably spiral guide groove (track) on the coating side advantageously has a groove depth of from 10 to 100 nm, preferably from 20 to 80 nm. The cross-sectional shape, periodic or quasi-periodic lateral deflection (wobble) as well as any additional markings between adjacent grooves (pre-pits) will be based on the HD-DVD™ type described above.

The reflector layer and the recording layer are applied to the substrate in that order. Either the grooves or the rail-like raised areas between them can be utilised as the track, reference usually being made to “in-groove” media in the first case and to “on-groove” media in the second case. Using the compounds of formula (I) it is advantageously possible to achieve both forms, possibly also simultaneously.

The recording medium is applied, for example, as indicated above, it being especially advantageous that it is possible also to select solvents that would attack the substrate material, for example chlorinated or aromatic hydrocarbons. The thickness of the layer, which is as amorphous as possible, can be uniform or it can be different in the grooves or on the raised portions. In the grooves, the thickness of the recording layer is advantageously from 20 to 200 nm, preferably from 30 to 150 nm, especially from 30 to 100 nm. When the track on the raised portions is to be used for recording, its layer thickness is advantageously from 10 to 120 nm, preferably from 20 to 100 nm, especially from 20 to 60 nm, whereas when only the groove is used as the track, a layer thickness of from 0 to 100 nm, preferably from 0 to 60 nm, especially from 0 to 20 nm, is sufficient. In both cases the track width (raised portions and/or indentations) is from 100 to 300 nm, preferably from 120 to 250 nm, especially from 150 to 200 nm, and the axial spacing between two tracks is from 200 to 600 nm, preferably from 250 to 400 nm, especially from 300 to 340 nm. Good results are obtained, for example, with a raised track (“on-groove”) 30±10 nm deep and 180±10 nm wide with an axial spacing of 320±10 nm. In that case the laser beam, with a high aperture, passes through the covering layer, which increases the resolution.

The inverse layer structure requires appreciably higher standards, however, which the compounds used according to the invention meet astonishingly well. Especially high standards are required, for example, when the recording layer is applied to the metallic reflector layer and especially when a covering layer is applied to the recording layer, the covering layer being required to provide the recording layer with adequate protection against abrasion, photo-oxidation, fingermarks, moisture and other environmental effects and advantageously having a thickness in the range from 0.01 to 0.5 mm, preferably in the range from 0.05 to 0.2 mm, especially in the range from 0.08 to 0.13 mm.

The covering layer preferably consists of a material that exhibits a transmission of 80% or above at the writing or readout wavelength of the laser. Suitable materials for the covering layer include, for example, those materials mentioned above, but especially polycarbonate (such as Pure Ace® or Panlite®, Teijin Ltd), cellulose triacetate (such as Fujitac®, Fuji Photo Film) or polyethylene terephthalate (such as Lumirror®, Toray Industry), special preference being given to polycarbonate. Especially in the case of directly applied covering layers, radiation-cured coatings, such as those already described above, are advantageous, for example SD 347™ (Dainippon Ink).

The covering layer can be applied directly to the solid recording layer by means of a suitable adhesion promoter. In another embodiment, there is applied to the solid recording layer an additional, thin separating layer of a metallic, crosslinked organometallic or preferably dielectric inorganic material, for example in a thickness of from 0.001 to 10 μm, preferably from 0.005 to 1 μm, especially from 0.01 to 0.1 μm, for example from 0.05 to 0.08 μm in the case of dielectric separating layers and from 0.01 to 0.03 μm in the case of metallic separating layers. Separating layers and corresponding methods are disclosed in WO 02/082438, to which reference is expressly made here. If desired, such coatings can be applied, for example, in the same thickness also between the support material and the metallic reflector layer or between the metallic reflector layer and the optical recording layer. This may be advantageous in certain cases, for example when a silver reflector is used in combination with sulfur-containing additives in the recording layer.

Analogously to the structure described above, it will be understood that, in this case too, the recording media can be assembled from two halves, two substrates being adhesively bonded before or after coating. It is in addition also possible to use a substrate that has grooves on both sides.

A very special advantage of the compounds according to the invention is the extraordinarily high modulation, especially in recording media corresponding to the Blu-ray™ standard. The compounds according to the invention can accordingly also be used to increase modulation in optical recording media.

In a recording medium having an inverse layer structure, the transparency of the substrate is, on the other hand, irrelevant. It is therefore also possible, for example, to use coloured (for example yellow, red, blue, green, white, grey or black pigmented) plastics or other synthetic or natural materials, such as steel, aluminium or other metals, or also paper (see Proceedings of SPIE Vol. 5380/04 “A 25 GB paper disk”).

The compounds of formula (I) used in accordance with the invention are novel. The invention therefore relates also to a compound of formula (I) according to the definition given hereinabove.

The compounds of formula (I) are advantageously prepared by reacting ligands with metal salts analogously to methods known per se. It is possible to use, for example, the process disclosed in WO 05/000 972.

Also novel are the compounds of formula

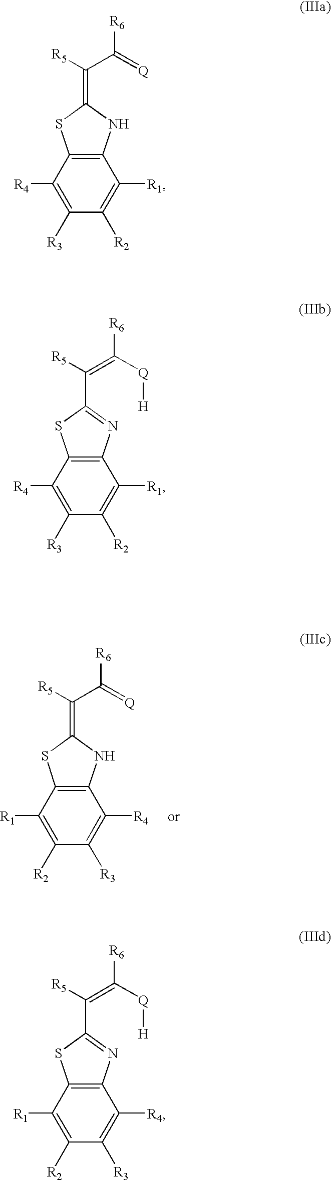

wherein R₆ is a C₁-C₅alkyl, C₂-C₅alkenyl, C₂-C₅alkynyl, C₃-C₅cycloalkyl, hetero-C₂-C₅cycloalkyl or C₃-C₅cycloalkenyl radical each of which is unsubstituted or is substituted according to the definitions in formula (I), with the proviso that there are no H atoms at the C atom by which that radical is bonded to the remainder of formula (IIIa) or (IIIb), and with the exception of the compound wherein R₆ is tert-butyl and R₅ is CN. R₅ is preferably H in formula (IIIa) or (IIIb). Especially preferred are unsubstituted or substituted tert-butyl, neopentyl and neopentenyl, and also perfluorinated C₁-C₅alkyl, especially CF₃ or C₂F₅.

Additional ligands, where present, are advantageously in 1 to 1.2 times the stoichiometric amount and are preferably not added until the last step of the preparation. The stoichiometric amount corresponds to the desired number of such ligands in the chelate of formula (I).

In some cases, precipitation of the desired product can be promoted or accelerated by diluting the mother liquor. Diluents can be selected according to customary criteria known per so in accordance with the reaction liquid and the polarity of the desired product; water or apolar hydrocarbons are frequently suitable.

Isolation of the chelates is generally effected by filtration or by extraction from the aqueous phase (where appropriate after addition of water) using a water-immiscible solvent. The isolation of substances by extraction by shaking, inclusive of all subsequent steps, are well known per se. It is, however, also possible for any other desired alternative method to be used, for example flash chromatography.

The following Examples illustrate the invention without limiting the scope thereof (unless otherwise indicated, “%” is always % by weight):

### EXAMPLE 1

1-Benzothiazol-2-yl-propan-2-one is prepared in accordance with Example 1 of U.S. Pat. No. 2,447,456 from 2-amino-thiophenol and diketene:

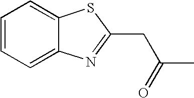

UV/VIS(CH₂Cl₂): λₘₐₓ=321; ε=4910 l·mol⁻¹·cm⁻¹.

### EXAMPLE 2

5.97 g of 2-methylbenzothiazole in 60 ml of abs. tetrahydrofuran (THF) are introduced into a thoroughly dried 250 ml multi-necked glass vessel equipped with a magnetic stirrer, thermometer, septum and nitrogen transfer line and cooled to −75° C. using a dry ice/ethanol bath. Using a syringe, 28 ml of a 1.6 M butyllithium solution in hexane are then added dropwise in the course of 55 minutes so that the internal temperature does not exceed −70° C. After stirring for 2 hours at −78° C., a solution of 10.66 g of trifluoroacetic acid ethyl ester in 40 ml of abs. THF is added dropwise to the resulting yellow suspension in the course of 45 minutes, so that the internal temperature does not exceed −70° C. Stirring is then carried out for 1 hour at −78° C., followed by heating to 23° C. and hydrolysis with 50 ml of saturated NaHCO₃ solution. The phases are separated and the aqueous phase is extracted 2× using 50 ml of ethyl acetate each time. The combined organic phases are washed 3× with saturated NaCl solution each time, dried over MgSO₄, filtered and concentrated by evaporation to 100 ml. The suspension which forms is filtered, and the residue is washed 2× using 20 ml of ice-cold ethyl acetate each time and dried at 40° C./5·10³ Pa. 1.98 g of 3-(3H-benzothiazol-2-ylidene)-1,1,1-trifluoropropan-2-one are obtained in the form of slightly yellow crystals:

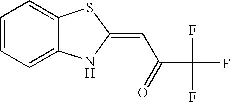

UV/VIS(CH₂Cl₂): λₘₐₓ=371; ε=23 200 l·mol⁻¹·cm⁻¹.

Further compounds are prepared analogously to Example 2 using phenyl-substituted 2-methyl-benzothiazoles as starting materials. Each of the starting materials can be prepared from the correspondingly substituted anilines according to methods known per se, for example by acetylation, conversion to thioacetamides and subsequent cyclisation to 2-methyl-benzothiazoles.

### EXAMPLE 3

### 1,1,1-Trifluoro-3-(5-methyl-3H-benzothiazol-2-ylidene)-propan-2-one

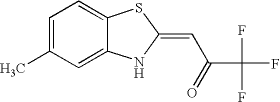 UV/VIS(CH₂Cl₂): λₘₐₓ=374; ε=22 500 l·mol⁻·cm⁻¹.

### EXAMPLE 4

### 1,1,1-Trifluoro-3-(5-methoxy-3H-benzothiazol-2-ylidene)-propan-2-one

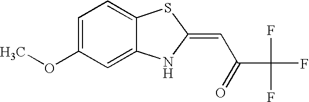 UV/VIS(CH₂Cl₂): λₘₐₓ=380; ε=21 900 l·mol⁻¹·cm⁻¹.

### EXAMPLE 5

### 3-(6-tert-Butyl-3H-benzothiazol-2-ylidene)-1,1,1-trifluoro-propan-2-one

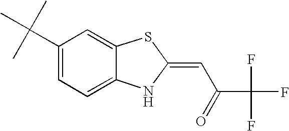 UV/VIS(N-methyl-pyrrolidone): λₘₐₓ=354; ε=33 700 l·mol⁻¹·cm⁻¹.

### EXAMPLE 6

### 1,1,1-Trifluoro-3-[6-isopropyl-3H-benzothiazol-2-ylidene]-propan-2-one

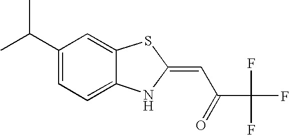 UV/VIS(N-methyl-pyrrolidone): λₘₐₓ=366 nm.

### EXAMPLE 7

### 1,1,1-Trifluoro-3-[6-methoxy-3H-benzothiazol-2-ylidene]-propan-2-one

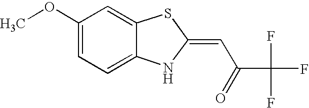 UV/VIS(CH₂Cl₂): λₘₐₓ=380 nm.

### EXAMPLE 8

420 mg of distilled 2-methylpyridine in 10 ml of abs. THF are introduced into a 50 ml multi-necked glass vessel equipped with a magnetic stirrer, thermometer, septum and nitrogen transfer line and, with stirring and under nitrogen, cooled to −50° C. using a dry ice/ethanol bath. In the course of 10 min., 2.5 ml of lithium diisopropylamide (LDA, 2M solution in THF/heptane/ethylbenzene) are added dropwise and the mixture is then heated to −10° C. 0.5 g of (3H-benzothiazol-2-ylidene)-acetic acid ethyl ester (prepared according to P. Baudet, Helv. Chim. Acta 53, 1683 [1970]) dissolved in 2 ml of abs. THF is then added dropwise in the course of 10 min. and stirring is carried out for 18 hours at 23° C. After the addition of 30 ml of water, extraction is carried out 3× using 50 ml of ethyl acetate each time. The combined organic phases are washed 2× with 10 ml of 2N HCl each time, and the combined HCl phases are adjusted to pH=8 using solid sodium hydrogen carbonate and extracted 3× with 50 ml of ethyl acetate each time. The combined organic phases are dried over MgSO₄, filtered and concentrated by evaporation, yielding 440 mg of crude 1-(3H-benzothiazol-2-ylidene)-3-pyridin-2-yl-propan-2-one, which is purified by flash chromatography on silica gel 60 using hexane/ethyl acetate (1:2) as eluant. 0.33 g of the yellow solid of formula:

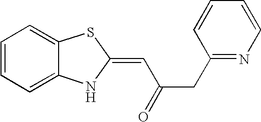

is obtained. UV/VIS(NMP): λₘₐₓ=362; ε=19 500 l·mol⁻¹·cm⁻¹.

### EXAMPLE 9

382 mg of 1-benzothiazol-2-yl-propan-2-one according to Example 1 are suspended in 5 ml of ethanol, and then 108 mg of sodium methanolate are added and stirring is carried out for 10 minutes at 23° C. A solution of 238 mg of cobalt(II) chloride hexahydrate in 3 ml of ethanol is added and stirring is carried out for a further 2 hours at 23° C. The precipitated product is filtered off and washed 3× with 1 ml of ethanol each time. Drying is carried out at 40° C./2.5·10³ Pa. 420 mg of bis(1-[3H-benzothiazol-2-ylidene]-propan-2-one)cobalt(II) are obtained in the form of an orange powder:

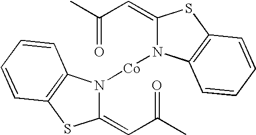

UV/VIS(CH₂Cl₂): λₘₐₓ=355; ε=39 100 l·mol⁻¹·cm⁻¹.

### EXAMPLE 10

382 mg of 1-benzothiazol-2-yl-propan-2-one according to Example 1 are suspended in 5 ml of ethanol, and then 108 mg of sodium methanolate are added and stirring is carried out for 10 minutes at 23° C. An ethanolic solution of 134 mg of copper(II) chloride is added and stirring is carried out for a further 2 hours at 23° C. The precipitated product is filtered off and washed three times with 1 ml of ethanol each time. Drying is carried out at 40° C./2.5·10³ Pa. 440 mg of bis(1-[3H-benzothiazol-2-ylidene]-propan-2-one)copper(II) are obtained in the form of a violet powder:

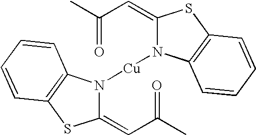

UV/VIS(CH₂Cl₂): λₘₐₓ=355; ε=31 500 l·mol⁻¹·cm⁻¹.

### EXAMPLE 11

382 mg of 1-benzothiazol-2-yl-propan-2-one according to Example 1 are suspended in 5 ml of ethanol, and then 108 mg of sodium methanolate are added and stirring is carried out for 10 minutes at 23° C. A solution of 130 mg of nickel(II) chloride in 1 ml of water is added and stirring is carried out for a further 2 hours at 23° C. The precipitated product is filtered off, washed 3× with 1 ml of ethanol each time and dried at 40° C./2.5·10³ Pa. 365 mg of bis(1-[3H-benzothiazol-2-ylidene]-propan-2-one)nickel(II) are obtained in the form of a yellowish green powder:

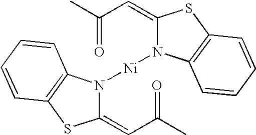

UV/VIS(CH₂Cl₂): λₘₐₓ=349; ε=35 100 l·mol⁻¹·cm⁻¹.

### EXAMPLE 12

4.06 g of the compound according to Example 4 are suspended in 70 ml of ethanol and then 7.37 ml of 2N sodium hydroxide solution are added and stirring is carried out for 10 minutes at 23° C. A solution of 1.75 g cobalt(II) chloride hexahydrate in 8 ml of ethanol is added and stirring is carried out for a further 20 hours at 23° C. The precipitated product is filtered off and washed three times with 20 ml of water each time. Drying is carried out at 40° C./2.5·10³ Pa. 3.99 g of bis(1,1,1-trifluoro-3-[5-methoxy-3H-benzothiazol-2-ylidene]-propan-2-one)cobalt(II) are obtained in the form of an orange powder:

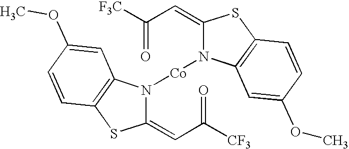

UV/VIS(CH₂Cl₂): λₘₐₓ=354; ε=39 200 l·mol⁻¹·cm⁻¹.

### EXAMPLE 13

100 mg of copper(II) acetate monohydrate are introduced into 5 ml of ethanol and stirred for 5 minutes. A clear light-blue solution is formed. There are added thereto 287 mg of the compound according to Example 6. A greenish blue precipitate is immediately formed. After 4 hours, filtration is carried out and the residue is washed with ethanol and dried at 20° C./2.5·10³ Pa. 265 mg of bis(1,1,1-trifluoro-3-[6-isopropyl-3H-benzothiazol-2-ylidene]-propan-2-one)copper(II) are obtained in the form of a violet powder:

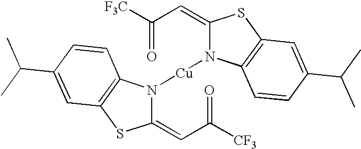

UV/VIS(CH₂Cl₂): λₘₐₓ=350; ε=41 100 l·mol⁻¹·cm⁻¹.

### EXAMPLE 14

124 mg of nickel(II) acetate tetrahydrate are introduced into 5 ml of ethanol and stirred for 10 min. A clear green solution is formed. There are added thereto 287 mg of the compound according to Example 6. After 24 hours, the solvent is concentrated by evaporation and the acetic acid formed is removed by azeotropic distillation 3× using 10 ml of toluene each time. The residue is dried at 40° C./2.5·10³ Pa. 315 mg of bis(1,1,1-trifluoro-3-[6-isopropyl-3H-benzothiazol-2-ylidene]-propan-2-one)nickel(II) are obtained in the form of a brown powder:

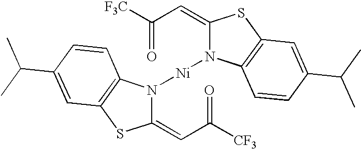

UV/VIS(CH₂Cl₂): λₘₐₓ=346; ε=36 600 l·mol⁻¹·cm⁻¹.

### EXAMPLE 15

1 g of cobalt(II) acetate tetrahydrate is introduced into 18 ml of ethanol and stirred for 10 minutes. A clear violet solution is formed. 1 g of the compound according to Example 6 is added thereto. An orange precipitate is immediately formed. After 24 hours, filtration is carried out and the residue is washed with ethanol and dried at 40° C./2.5·10³ Pa. 928 mg of bis(1,1,1-trifluoro-3-[6-isopropyl-3H-benzothiazol-2-ylidene]-propan-2-one)cobalt(II) are obtained in the form of an orange powder:

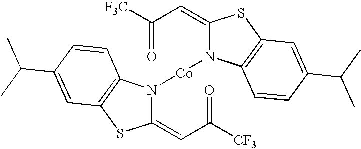

UV/VIS(CH₂Cl₂): λₘₐₓ=343; ε=45 400 l·mol⁻¹·cm⁻¹.

### EXAMPLE 16

Proceeding analogously to Example 12, but using the compound according to Example 7 instead of the compound according to Example 4, bis(1,1,1-trifluoro-3-[6-methoxy-3H-benzothiazol-2-ylidene]-propan-2-one)cobalt(II) is obtained in a yield of 89% in the form of an orange powder:

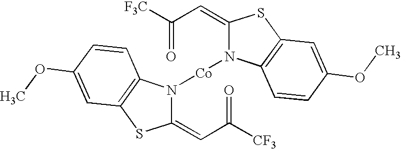

UV/VIS(CH₂Cl₂): λₘₐₓ=349; ε=43 300 l·mol⁻¹·cm⁻¹.

### EXAMPLE 17

Proceeding analogously to Example 12, but using equivalent amounts of the compounds according to Examples 6 and 7 instead of solely the compound according to Example 4, a yellowish orange pulverulent mixture of the compounds according to Examples 15 and 16 and (1,1,1-trifluoro-3-[6-methoxy-3H-benzothiazol-2-ylidene]-propan-2-one)-(1,1,1-trifluoro-3-[6-isopropyl-3H-benzothiazol-2-ylidene]propan-2-one)cobalt(II) is obtained in a good yield:

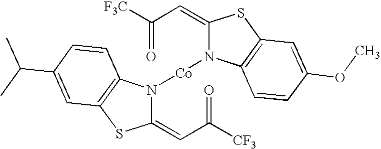

UV/VIS(CH₂Cl₂): λₘₐₓ=346; ε=43 700 l·mol⁻¹·cm⁻¹.

The asymmetric compound can optionally be obtained in pure form by chromatography.

### EXAMPLE 18

Proceeding analogously to Example 17, but using an equivalent amount of copper(II) acetate monohydrate instead of cobalt(II) chloride hexahydrate, there is obtained in 90% yield a violet powder containing the compound of the following formula:

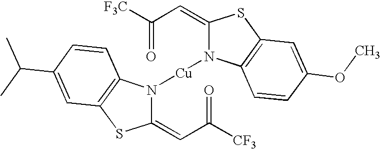

UV/VIS(CH₂Cl₂): λₘₐₓ=350; ε=42 700 l·mol⁻¹·cm⁻¹.

### EXAMPLE 19

Proceeding analogously to Example 15, but using a 4:1-molar mixture of the compounds according to Examples 5 and 7 instead of solely the compound according to Example 4, there is obtained an orange, pulverulent mixture of the compounds of formulae

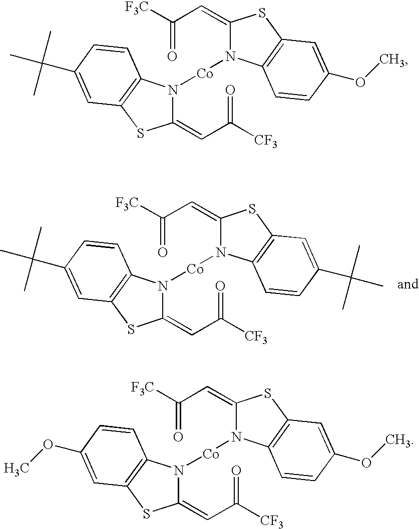

UV/VIS(CH₂Cl₂): λₘₐₓ=349; ε=44 600 l·mol⁻¹·cm⁻.

The individual components of the mixture can optionally be obtained in pure form by chromatography.

### EXAMPLE 20

7.91 g of 5-bromo-2-methyl-benzothiazole in 550 ml of dry dimethyl-formamide (DMF) are introduced into a 1 litre multi-necked flask equipped with a magnetic stirrer, dropping funnel, thermometer, reflux condenser and nitrogen transfer line, 520 mg of palladium chloride and 520 mg of triphenylphosphine are added and, with stirring and under nitrogen, heating is carried out at 140° C. In the course of 30 min., 47.5 g of triethyl phosphite are added dropwise and then stirring is carried out for 24 hours at the same temperature. The mixture is then cooled to 23° C., poured into 1500 ml of water and extracted 3× using 300 ml of ethyl acetate each time. The combined organic phases are washed 3× with 500 ml of water each time, dried over magnesium sulfate, filtered and concentrated by evaporation. The residue is purified by flash chromatography (eluant hexane/ethyl acetate (1:6)). (2-Methyl-benzothiazol-5-yl)-phosphonic acid diethyl ester is obtained in a yield of 90% in the form of a slightly yellow oil:

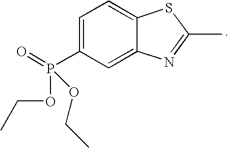

### EXAMPLE 21

19.4 g of 2-methyl-6-nitro-benzothiazole and 13.77 g of powdered iron in 250 ml of acetic acid are introduced into a 750 ml multi-necked flask equipped with an anchor stirrer, thermometer, reflux condenser and nitrogen transfer line, and refluxed for 1 hour, with stirring and under nitrogen. The mixture is then cooled to 23° C., 200 ml of acetic anhydride are added and stirring is carried out for 30 min. at 23° C. The reaction mixture is filtered and the residue is then washed 2× with 50 ml of acetic acid each time. The filtrate is added dropwise, with stirring and cooling, to 1 litre of 40% sodium hydroxide solution, and the precipitate is filtered off and washed thoroughly with water. The residue is dissolved in 1 litre of acetone, filtered and the filtrate is concentrated by evaporation. The crude product is dissolved in 200 ml of toluene at reflux, undissolved components are decanted off, and refluxing is carried out again, followed by cooling slowly to 23° C., and then to 0° C. The suspension is filtered, and the residue is washed 2× with 10 ml of ice-cold toluene each time and dried for 18 hours at 50° C./2.5·10³ Pa. Slightly beige crystals of N-(2-methyl-benzothiazol-6-yl)-acetamide are obtained in a yield of 82%:

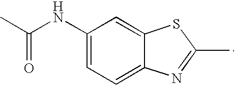

### EXAMPLE 22

8.8 g of 6-amino-2-methyl-benzothiazole and 14.0 g of di-tert-butyl dicarbonate in 300 ml of toluene are introduced into a 500 ml multi-necked flask equipped with a magnetic stirrer, thermometer, reflux condenser and nitrogen transfer line and refluxed for 24 hours, with stirring and under nitrogen. Distilling off to 250 ml of toluene is then carried out, 250 ml of hexane are added dropwise, and the resulting solution is cooled, with stirring, first to 23° C. and then to 0° C. The suspension is filtered, and the residue is washed 3× with 50 ml of hexane each time and dried for 18 hours at 40° C./2.5·10³ Pa. White crystals of (2-methylbenzothiazol-6-yl)-carbamoyl-tert-butyl ester are obtained in a yield of 89%:

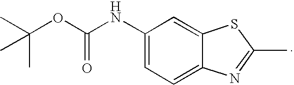

### EXAMPLE 23

Using diisopropyl dicarbonate instead of di-tert-butyl dicarbonate, (2-methyl-benzothiazol-6-yl)-carbamoyl-isopropyl ester is obtained analogously to Example 22 in a yield of 65% in the form of a beige solid:

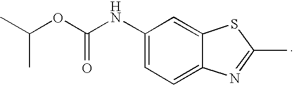

### EXAMPLE 24

Using diethyl pyrocarbonate instead of di-tert-butyl dicarbonate, (2-methyl-benzothiazol-6-yl)-carbamoyl-ethyl ester is obtained analogously to Example 22 in a yield of 85% in the form of a white solid:

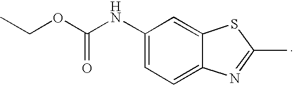

### EXAMPLE 25

1 g of 6-amino-2-methyl-benzothiazole is introduced into 30 ml of dichloromethane in a 100 ml multi-necked flask equipped with a magnetic stirrer, thermometer and nitrogen transfer line, and the clear yellow solution is cooled to −65° C. with stirring. In the course of 30 min., 3.51 g of trifluoromethanesulfonic anhydride are added dropwise and the milky reaction mixture is stirred for 3 hours at −65° C. and then for 1 hour at 23° C. It is then poured into 50 ml of ice-water, the phases are separated and the aqueous phase is extracted 2× using 30 ml of dichloromethane each time. The combined organic phases are washed 3× with each time 100 ml of water and with 50 ml of phosphate buffer (pH=5), dried over MgSO₄, filtered and concentrated by evaporation. 1.75 g of a brown residue are obtained, which is purified by flash chromatography using hexane/ethyl acetate (1:1) as eluant. Trifluoro-N-(2-methyl-benzothiazol-6-yl)-methanesulfonamide is obtained in a yield of 67% in the form of an orange solid:

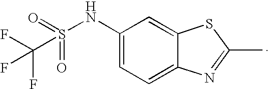

### EXAMPLE 26

Using methanesulfonyl chloride instead of trifluoromethanesulfonic anhydride, N-(2-methyl-benzothiazol-6-yl)-methanesulfonamide is obtained analogously to Example 25 in a yield of 89% in the form of a beige solid:

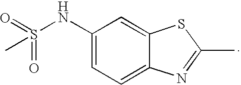

### EXAMPLE 27

2.1 g of 2,5-diamino-1,4-benzodithiol-dihydrochloride in 20 ml of water are introduced into a 50 ml multi-necked flask equipped with a magnetic stirrer, thermometer, reflux condenser and nitrogen transfer line. With stirring and under nitrogen, 16.3 ml of 1N aqueous NaOH solution are added dropwise. 3.33 g of acetic anhydride are then added and the suspension is refluxed for 18 hours. The suspension is then cooled and extracted 3× using 30 ml of ethyl acetate each time, and the combined organic phases are washed 3× with 50 ml of water each time, dried over magnesium sulfate, filtered and concentrated by evaporation. 2,6-Dimethyl-benzo[1,2-d:4,5-d]bisthiazole is obtained in the form of a yellow solid in a yield of 74%:

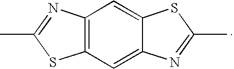

### EXAMPLE 28

1.16 g of oxalyl chloride are weighed into a 50 ml multi-necked flask equipped with a magnetic stirrer, thermometer, dropping funnel and nitrogen transfer line and 10 ml of 1,4-dioxane are added. With stirring, cooling to 5° C. is carried out and, in the course of 15 min., a mixture of 3 g of 6-amino-2-methylbenzothiazole and 1.85 g of triethylamine, dissolved in 60 ml of 1,4-dioxane, is slowly added dropwise. A thick suspension is formed which, after removal of the ice bath, becomes readily stirrable again. After 4 hours at 23° C., the suspension is poured into 500 ml of water, stirred for 30 min. and filtered, and the residue is washed thoroughly with water and dried for 18 hours at 40° C./2.5·10³ Pa. N,N′-Bis(2-methyl-benzothiazol-6-yl)-oxamide is obtained in a yield of 69% in the form of beige crystals:

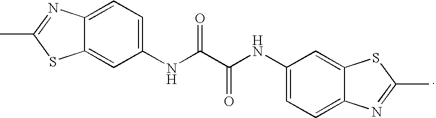

### EXAMPLE 29

19.19 g of 4-aminobenzoic acid ethyl ester and 22.58 g of potassium thiocyanate in 170 ml of acetic acid are introduced into a 500 ml multi-necked flask equipped with a magnetic stirrer, thermometer, reflux condenser, dropping funnel and nitrogen transfer line and heated at 40° C. until a clear solution is obtained. The solution is then cooled to 10° C. and, in the course of 1 hour, 18.47 g of bromine dissolved in 30 ml of acetic acid are added dropwise so that the internal temperature does not exceed 15° C. A yellow suspension is formed, which is stirred for a further 2 hours at the same temperature and then stirred for 18 hours at 23° C. 40 ml of a saturated aqueous sodium thiosulfate solution are subsequently added dropwise, filtration is carried out, and the residue is then washed with 150 ml of acetic acid. The mother liquor is cooled using an ice bath and, with stirring, adjusted to pH 10 using 4N aqueous NaOH solution. The suspension is then filtered and the residue is washed 4× with 250 ml of cold water each time and dried for 18 hours at 50° C./2.5·10³ Pa. 2-Amino-benzothiazole-6-carboxylic acid ethyl ester is obtained in a yield of 77% in the form of a yellow solid.

In a 100 ml multi-necked flask equipped with a blade stirrer, thermometer, reflux condenser, gas inlet tube and nitrogen transfer line, 6.7 g of the intermediate product are refluxed for 26 hours with 4 equivalents of a 6.7N aqueous potassium hydroxide solution, with vigorous stirring and under a stream of nitrogen, until the formation of ammonia can no longer be detected. The yellow solution is then diluted with 10 ml of water and adjusted to pH 3 using 40% sulfuric acid. To the yellow suspension that forms, there are added dropwise, in the course of 5 min., 6.19 g of acetic anhydride. The reaction mixture is then refluxed for 18 hours and subsequently cooled to 23° C. After the addition of 50 ml of ethyl acetate, the phases are separated and the aqueous phase is extracted 2× using 75 ml of ethyl acetate each time. The combined organic phases are washed 3× with 150 ml of water each time, dried over magnesium sulfate, filtered and concentrated by evaporation. 2-Methyl-benzothiazole-6-carboxylic acid is obtained in a yield of 45% in the form of a beige solid.

2.6 g of the intermediate product are suspended in 60 ml of ethanol and, in the course of 10 min., added dropwise to a solution of 15 ml of concentrated sulfuric acid in 60 ml of ethanol in a 250 ml multi-necked flask equipped with a magnetic stirrer, thermometer, dropping funnel, reflux condenser and nitrogen transfer line. Refluxing is then carried out for 18 hours with stirring and under nitrogen. The clear orange solution is cooled to 23° C. and adjusted to pH 10 using a 2N aqueous NaOH solution. 100 ml of water are added and extraction is carried out 3× using 150 ml of ethyl acetate each time. The combined organic phases are washed 2× with 100 ml of water each time, dried over magnesium sulfate, filtered and concentrated by evaporation. The crude product is purified by flash chromatography using hexane/ethyl acetate (4:1) as eluant. 0.63 g of 2-methylbenzothiazole-6-carboxylic acid ethyl ester is obtained in the form of a yellow oil:

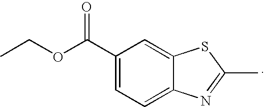

### EXAMPLE 30

7.2 g of ferrocenecarboxylic acid chloride are dissolved in 125 ml of pyridine in a 500 ml multi-necked flask equipped with a magnetic stirrer, thermometer and nitrogen transfer line, 190 mg of dimethylaminopyridine and 2.46 g of 6-amino-2-methyl-benzothiazole are added and stirring is carried out for 3 hours at 23° C. The reaction mixture is then poured, with stirring, into 1.2 litres of water, stirring is carried out for 1 hour, and the precipitate is filtered off and washed thoroughly with water and dried for 18 hours at 50° C./2.5·10³ Pa. The crude product is purified by flash chromatography (eluant:hexane/ethyl acetate (2:1)). (2-Methyl-benzothiazol-6-yl)-ferrocenylcarboxylic acid amide is obtained in a yield of 67% in the form of an orange solid:

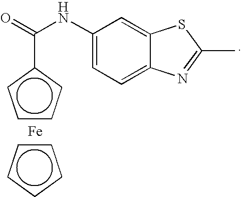

### EXAMPLE 31

1 g of 6-amino-2-methyl-benzothiazole are dissolved in 10 ml of pyridine in a 25 ml multi-necked flask equipped with a magnetic stirrer, thermometer and nitrogen transfer line, 0.93 g of tert-butyl isocyanate is added dropwise and stirring is carried out for 24 hours at 23° C. The reaction mixture is then added dropwise, with stirring, to 150 ml of ice-water and subsequently stirred for 30 min.; the precipitate is filtered off, washed thoroughly with water and dried for 18 hours at 50° C./2.5·10³ Pa. 1-tert-Butyl-3-(2-methyl-benzothiazol-6-yl)-urea is obtained in a yield of 87% in the form of a white solid:

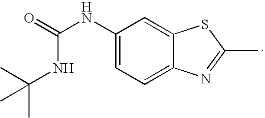

### EXAMPLE 32

4.0 g of 6-methoxy-2-methyl-benzothiazole and 6.37 g of triethyloxonium tetrafluoroborate in 35 ml of 1,2-dichloroethane are introduced into a 50 ml multi-necked flask equipped with a magnetic stirrer, thermometer and nitrogen transfer line, and the clear solution is stirred for 24 hours under nitrogen. The white suspension is then filtered and the residue is washed 2× with 10 ml of 1,2-dichloroethane each time and dried for 18 hours at 40° C./2.5·10³ Pa. White crystals of 3-ethyl-6-methoxy-2-methyl-benzothiazol-3-ium tetrafluoroborate are obtained in a yield of 55%.

1.56 g of that product are dissolved in 20 ml of dry pyridine in a 50 ml multi-necked flask equipped with a magnetic stirrer, thermometer, septum, reflux condenser and nitrogen transfer line; the solution is cooled to 0° C., with stirring and under a nitrogen atmosphere, and 0.75 g of acetyl chloride is added dropwise in the course of 10 min. Rapid heating to an internal temperature of 100° C. is then carried out using a preheated oil bath, and that temperature is maintained for 10 min. Rapid cooling, and filtration, are then carried out and the residue is washed with 75 ml of ethyl acetate. The filtrate is washed 4× with 25 ml of saturated aqueous NaCl solution each time, dried over magnesium sulfate, filtered and concentrated by evaporation. The residue is purified by flash chromatography (eluant hexane/ethyl acetate (1:2)). 1-[3-Ethyl-6-methoxy-3H-benzothiazol-2-ylidene]-propan-2-one is obtained in a yield of 37% in the form of a slightly yellow solid:

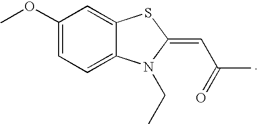

### EXAMPLE 33

Analogously to Example 2, 3-[6-ethoxy-3H-benzothiazol-(2)-ylidene]-1,1,1-trifluoro-propan-2-one is obtained in a yield of 58%:

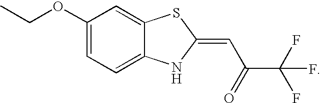

### EXAMPLE 34

2.81 g of potassium tert-butanoate in 30 ml of abs. THF are introduced into a 100 ml multi-necked flask equipped with a magnetic stirrer, thermometer, septum and nitrogen transfer line and, with stirring and under nitrogen, cooled to 3° C. using an ice bath. In the course of 45 min., 1.94 g of 4-methoxy-2-methyl-benzothiazole and 2.84 g of trifluoroacetic acid ethyl ester, dissolved in 40 ml of abs. THF, are so added dropwise that the internal temperature does not exceed 5° C. Stirring is then carried out for 1 hour at 0° C., the ice bath is subsequently removed, and the reaction mixture is stirred for 18 hours at 23° C. Hydrolysis is then carried out using 30 ml of 10% aqueous citric acid and the mixture is poured, with stirring, into 700 ml of water and stirred for 1 hour. Filtration is then carried out, followed by washing 3× with 100 ml of water each time, and the residue is dried for 18 hours at 50° C./2.5·10³ Pa. 1.73 g of 1,1,1-trifluoro-3-[4-methoxy-3H-benzothiazol-(2)-ylidene]-propan-2-one are obtained in the form of a slightly yellow solid:

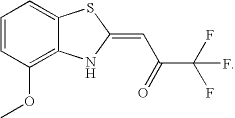

### EXAMPLES 35-54

The following compounds are prepared analogously to Example 34:

| Example | Structure | Yield |
|---|---|---|
| 35 | 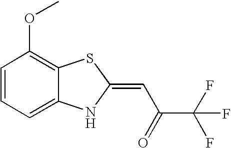 | 89% |
| 36 | 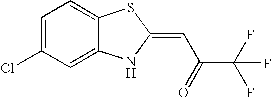 | 68% |
| 37 | 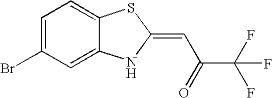 | 67% |
| 38 | 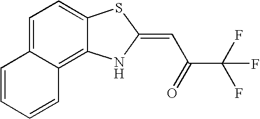 | 21% |
| 39 | 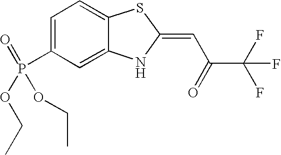 | 30% |
| 40 | 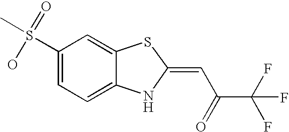 | 71% |
| 41 | 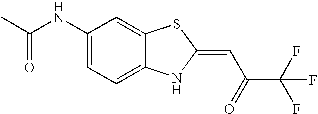 | 86% |
| 42 | 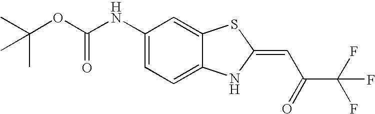 | 86% |
| 43 | 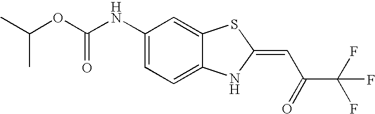 | 83% |
| 44 | 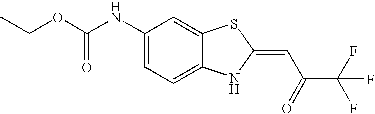 | 81% |
| 45 | 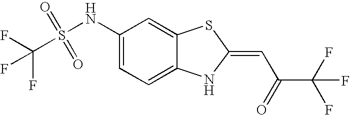 | 62% |
| 46 | 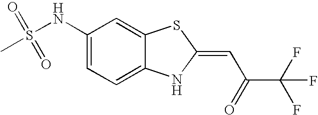 | 57% |
| 47 | 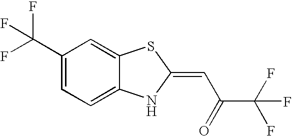 | 67% |
| 48 | 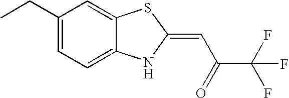 | 83% |
| 49 | 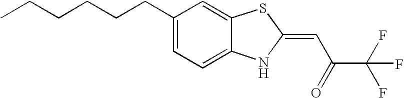 | 17% |
| 50 | 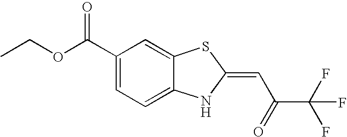 | 47% |
| 51 | 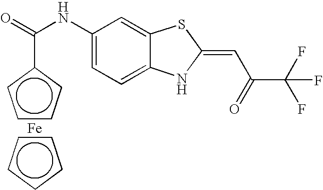 | 91% |
| 52 | 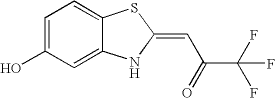 | 79% |
| 53 | 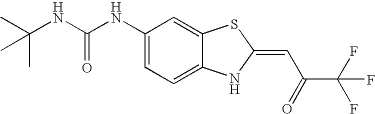 | 30% |
| 54 | 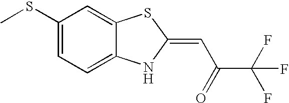 | 92% |

### EXAMPLE 55

3,3-Difluoro-1,5-bis[6-methoxy-3H-benzothiazol-2-ylidene]-pentane-2,4-dione is prepared analogously to Example 2, using two equivalents of 6-methoxy-2-methylbenzothiazole and 1 equivalent of diethyl difluoromalonate. The compound of the following structure is obtained in a yield of 40%:

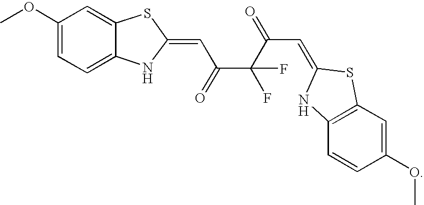

### EXAMPLE 56

The compound according to Example 27 is reacted analogously to Example 34 with 3.8 equivalents of trifluoroacetic acid ethyl ester and 4.6 equivalents of potassium tert-butanoate. 1,1,1-Trifluoro-3-[6-[3,3,3-trifluoro-2-oxo-prop-2-ylidene]-6,7-dihydro-3H-benzo[1,2-d; 4,5-d′]bisthiazol-2-ylidene]-propan-2-one is obtained in a yield of 25%:

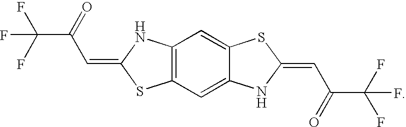

### EXAMPLE 57

Using ethyl acetate instead of trifluoroacetic acid ethyl ester, 1-[6-[2-oxo-prop-2-ylidene]-6,7-dihydro-3H-benz[1,2-d:4,5-d′]bisthiazol-2-ylidene]-propan-2-one is obtained analogously to Example 56 in a yield of 31%:

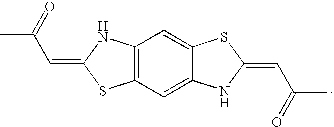

### EXAMPLE 58

1,1,1-Trifluoro-3-[4-methyl-6-{4-methyl-2-[3,3,3-trifluoro-2-oxo-propylidene]-2,3-dihydro-benzothiazol-6-ylmethyl}-3H-benzothiazol-2-ylidene]-propan-2-one is obtained analogously to Example 56 in a yield of 64%:

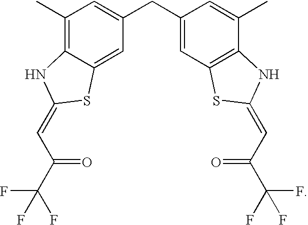

### EXAMPLE 59

The compound from Example 28 is reacted analogously to Example 34 with 4 equivalents of trifluoroacetic acid ethyl ester and 8 equivalents of potassium tert-butanoate. N,N′-Bis{2-[3,3,3-trifluoro-2-oxo-propylidene]-2,3-dihydro-benzothiazol-6-yl}-oxamide is obtained in a yield of 91%:

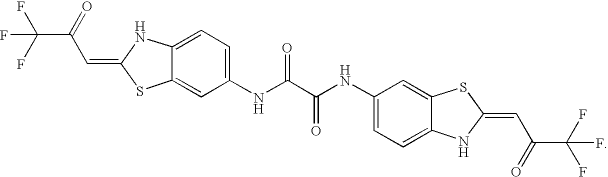

### EXAMPLE 60

Using benzoic acid ethyl ester instead of trifluoroacetic acid ethyl ester, 2-[6-methoxy-3H-benzothiazol-2-ylidene]-1-phenyl-ethanone is obtained analogously to Example 34 in a yield of 90%:

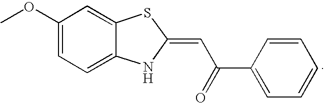

### EXAMPLE 61

Using pentafluoropropionic acid ethyl ester instead of trifluoroacetic acid ethyl ester, 3,3,4,4,4-pentafluoro-1-[6-methoxy-3H-benzothiazol-2-ylidene]-butan-2-one is obtained analogously to Example 34 in a yield of 85%:

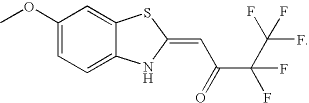

### EXAMPLE 62

Using pivalic acid ethyl ester instead of trifluoroacetic acid ethyl ester, 1-[6-methoxy-3H-benzothiazol-2-ylidene]-3,3-dimethyl-butan-2-one is obtained analogously to Example 2 in a yield of 28%:

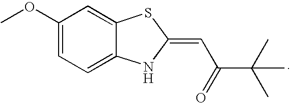

### EXAMPLE 63

Using 6-amino-2-methyl-benzothiazole, 4 equivalents of trifluoroacetic acid ethyl ester and 5 equivalents of potassium tert-butanoate, 2,2,2-trifluoro-N-{2-[3,3,3-trifluoro-2-oxo-propylidene]-2,3-dihydro-benzothiazol-6-yl}-acetamide is obtained analogously to Example 34 in a yield of 97%:

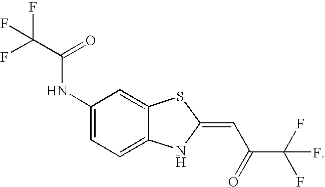

### EXAMPLE 64

In a 500 ml multi-necked flask equipped with a magnetic stirrer, thermometer, reflux condenser and nitrogen transfer line, 5 g of the compound according to Example 42 are introduced into 200 ml of 1,4-dioxane and 40 ml of 2N aqueous H₂SO₄, and the yellow suspension is refluxed with stirring, a clear yellow solution being formed. After 4 hours, the solution is cooled to 23° C., diluted with 1 litre of water and extracted 3× using 250 ml of ethyl acetate (EtOAc) each time. The combined organic phases are washed 3× with 100 ml of water each time, dried over MgSO₄, filtered and concentrated by evaporation. 1.9 g of a yellow solid are obtained, which is stirred at reflux for 1 hour in 50 ml of EtOAc. The suspension is then cooled, with stirring, to 0° C., filtered, washed 2× with 10 ml of ice-cold EtOAc each time, and dried for 18 hours at 50° C./2.5·10³ Pa. 0.94 g of 3-[6-amino-3H-benzothiazol-2-ylidene]-1,1,1-trifluoro-propan-2-one is obtained in the form of a yellow solid:

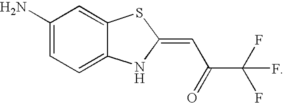

### EXAMPLE 65

1.75 g of the compound from Example 52, 1.79 g of di-tert-butyl dicarbonate, 0.17 g of dimethylaminopyridine and 20 ml of acetonitrile are introduced into a 50 ml multi-necked flask equipped with a magnetic stirrer, thermometer and nitrogen transfer line, and the yellow suspension is stirred for 18 hours. 20 ml of 1N hydrochloric acid are then added dropwise, stirring is carried out for 30 min., followed by filtration and washing 3× with 10 ml of water each time. The residue is dried for 18 hours at 50° C./2.5·10³ Pa. 1.7 g of a beige solid is obtained, which is recrystallised from 25 ml of isopropanol. 0.96 g of carbonic acid tert-butyl ester 2-[3,3,3-trifluoro-2-oxo-propylidene]-2,3-dihydro-benzothiazol-6-yl ester is obtained in the form of a light-yellow solid:

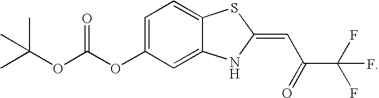

### EXAMPLE 66

Using tert-butyl isocyanate instead of di-tert-butyl dicarbonate, tertbutyl-carbamic acid 2-[3,3,3-trifluoro-2-oxo-propylidene]-2,3-dihydro-benzothiazol-5-yl ester is obtained analogously to Example 65 in a yield of 43% in the form of a yellow solid:

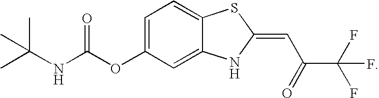

### EXAMPLES 67-85

The following complexes are prepared analogously to Example 12:

| | Ligand | | λₘₐₓ | ε | |
|---|---|---|---|---|---|
| Ex. | from Ex. | Structure | [nm] | [l · mol⁻¹ · cm⁻¹] | Solv. |
| 67 | 41 | 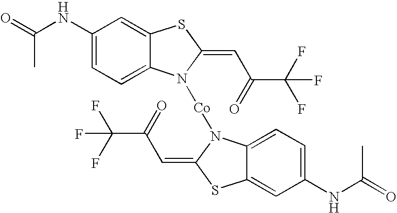 | 350 | 46500 | ethanol |
| 68 | 42 | 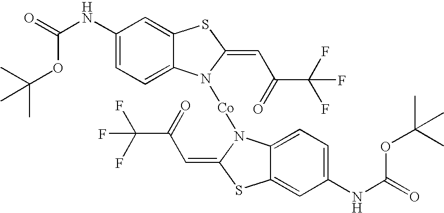 | 353 | 48900 | CH₂Cl₂ |
| 69 | 43 | 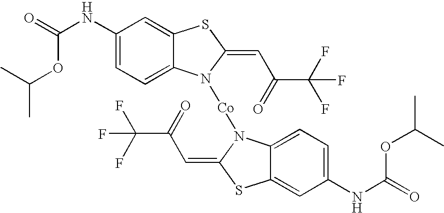 | 353 | 45000 | CH₂Cl₂ |
| 70 | 44 | 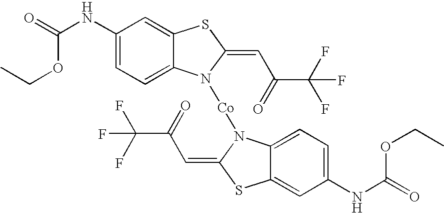 | 352 | 31900 | CH₂Cl₂ |
| 71 | 62 | 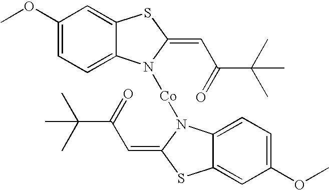 | 351 | 48700 | CH₂Cl₂ |
| 72 | 47 | 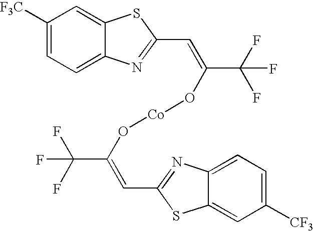 | 343 | 42000 | CH₂Cl₂ |
| 73 | 48 | 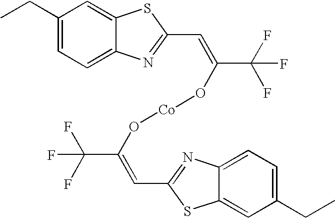 | 343 | 39800 | CH₂Cl₂ |
| 74 | 49 | 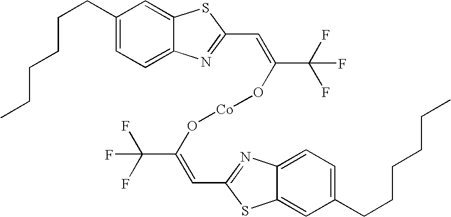 | 344 | 43600 | CH₂Cl₂ |
| 75 | 50 | 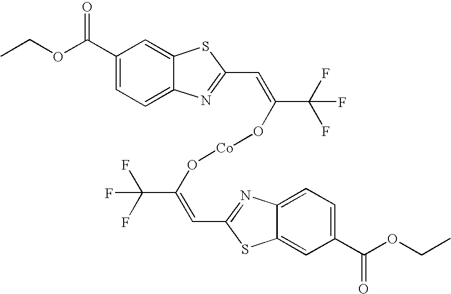 | 351 | 46200 | CH₂Cl₂ |
| 76 | 63 | 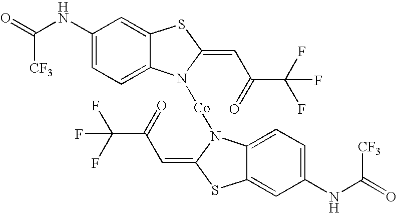 | 351 | 46600 | ethanol |
| 77 | 64 | 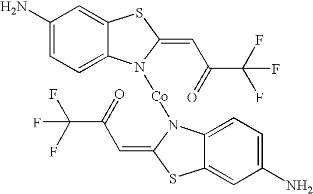 | 367 | 33300 | DMF |
| 78 | 45 | 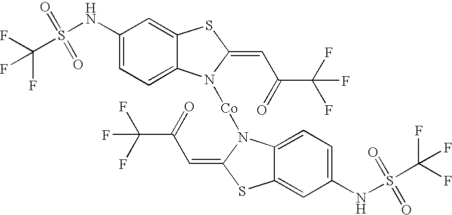 | 372 | 35800 | CH₂Cl₂ |
| 79 | 46 | 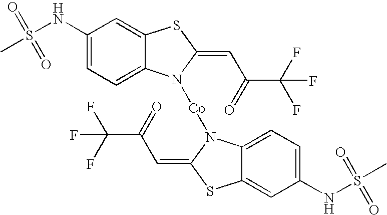 | 346 | 39600 | ethanol |
| 80 | 51 | 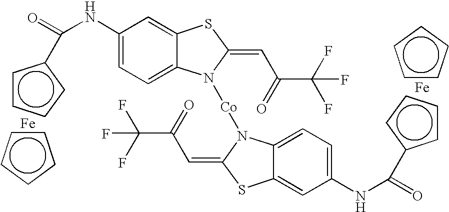 | 364 | 52300 | ethanol |
| 81 | 52 | 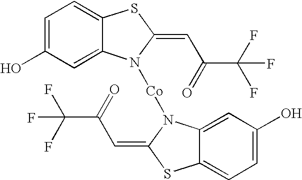 | 352 | 34300 | CH₂Cl₂ |
| 82 | 53 | 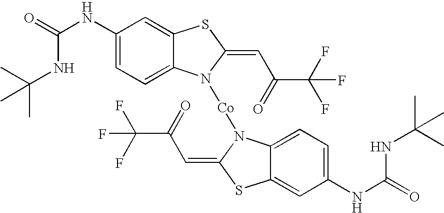 | 361 | 48200 | CH₂Cl₂ |
| 83 | 65 | 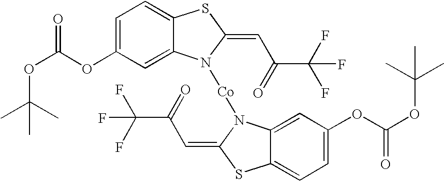 | 344 | 40300 | CH₂Cl₂ |
| 84 | 66 | 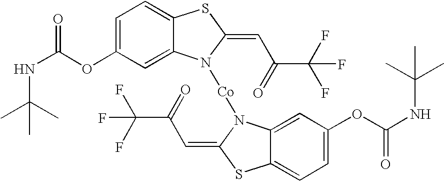 | 347 | 42100 | CH₂Cl₂ |
| 85 | 54 | 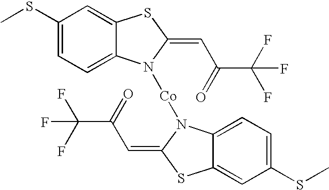 | 359 | 48100 | CH₂Cl₂ |

### EXAMPLES 86-96

The following complexes are prepared analogously to Example 15

| | Ligand | | λₘₐₓ | ε | |
|---|---|---|---|---|---|
| Ex. | from Ex. | Structure | [nm] | [l · mol⁻¹ · cm⁻¹] | Solv. |
| 86 | 5 | 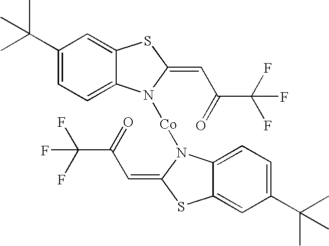 | 343 | 47100 | CH₂Cl₂ |
| 87 | 33 | 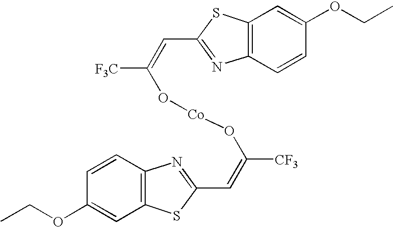 | 349 | 43400 | CH₂Cl₂ |
| 88 | 60 | 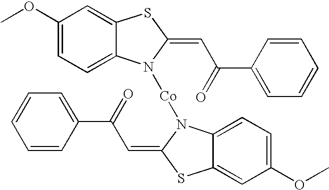 | 381 | 59500 | CH₂Cl₂ |
| 89 | 34 | 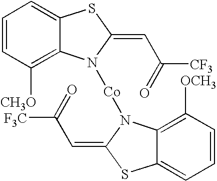 | 344 | 38300 | CH₂Cl₂ |
| 90 | 35 | 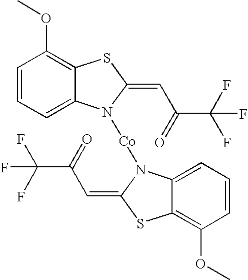 | 340 | 40100 | CH₂Cl₂ |
| 91 | 36 | 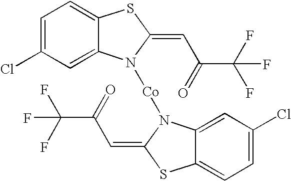 | 346 | 39700 | CH₂Cl₂ |
| 92 | 37 | 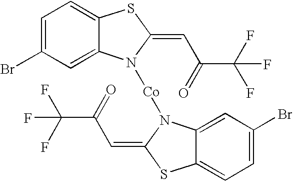 | 346 | 39700 | CH₂Cl₂ |
| 93 | 61 | 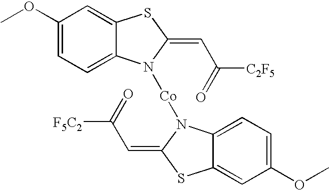 | 351 | 42200 | CH₂Cl₂ |
| 94 | 38 | 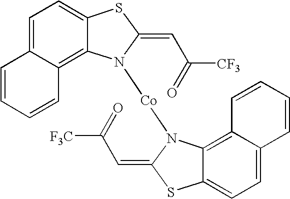 | 362 | 38800 | CH₂Cl₂ |
| 95 | 39 | 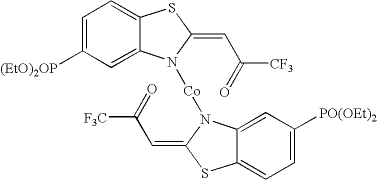 | 341 | 35200 | CH₂Cl₂ |
| 96 | 40 | 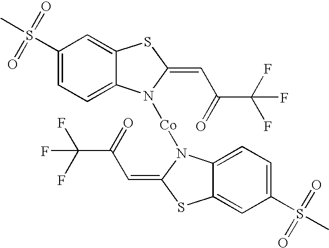 | 349 | 40700 | CH₂Cl₂ |

### EXAMPLE 97

The following complex is prepared analogously to Example 15, using Pd(II) acetate instead of Co(II) acetate:

| | Ligand | | λₘₐₓ | ε | |
|---|---|---|---|---|---|
| Ex. | from Ex. | Structure | [nm] | [l · mol⁻¹ · cm⁻¹] | Solv. |
| 97 | 4 | 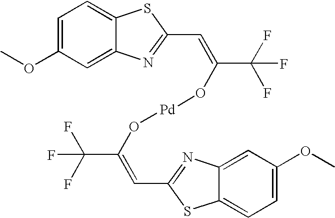 | 357 | 32800 | CH₂Cl₂ |

### EXAMPLE 98

The following complex is prepared analogously to Example 13:

| | | | λₘₐₓ | ε | |
|---|---|---|---|---|---|
| Ex. | Ligand | Structure | [nm] | [l · mol⁻¹ · cm⁻¹] | Solv. |
| 98 | BeilsteinReg. No.7030012 | 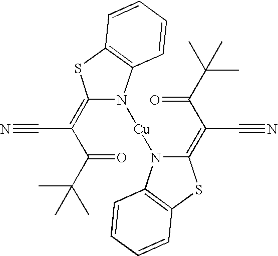 | 360 | 41000 | CH₂Cl₂ |

### EXAMPLES 99-106

Proceeding analogously to Example 15, but using equivalent amounts of the compounds listed in the following Table, mixtures of compounds as shown hereinbelow are obtained.

| | Ligands | | λₘₐₓ | ε | |
|---|---|---|---|---|---|
| Ex. | from Ex. | Structure | [nm] | [l · mol⁻¹ · cm⁻¹] | Solv. |
| 99 | 4 + 35 | 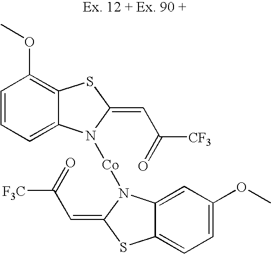 | 351 | 37200 | CH₂Cl₂ |
| 100 | 4 + 34 | 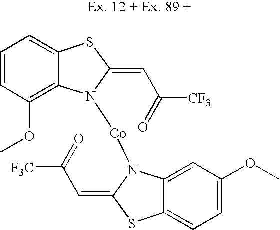 | 348 | 39200 | CH₂Cl₂ |
| 101 | 4 + 36 | 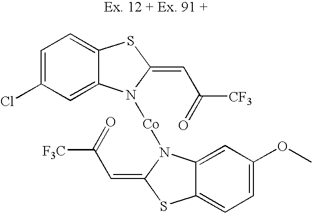 | 354 | 39500 | CH₂Cl₂ |
| 102 | 4 + 37 | 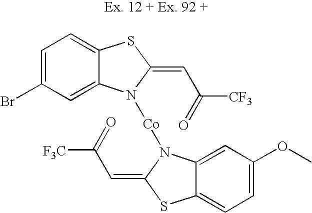 | 354 | 39500 | CH₂Cl₂ |
| 103 | 4 + 61 | 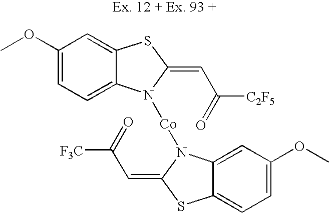 | 352 | 38300 | CH₂Cl₂ |
| 104 | 4 + 38 | 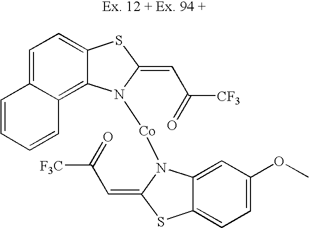 | 361 | 37600 | CH₂Cl₂ |
| 105 | 4 + 5 | 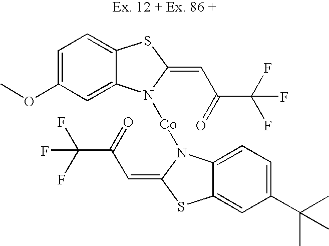 | 349 | 40200 | CH₂Cl₂ |
| 106 | 4 + 7 | 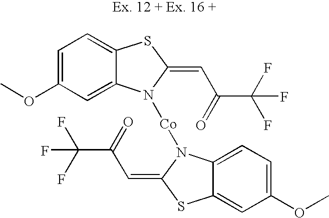 | 352 | 41600 | CH₂Cl₂ |

### EXAMPLE 107

Proceeding analogously to Example 17, but using equimolar amounts of the ligands from Example 4, Example 7, Example 34 and Example 35, a mixture of complexes is obtained that comprises, inter alia, complexes of Example 99, Example 100 and Example 106.

UV/VIS(CH₂Cl₂): λₘₐₓ=347 nm; ε=39 400 l·mol⁻¹·cm⁻¹.

### EXAMPLES 108-126

Proceeding analogously to Example 17, but using the compounds listed in the following Table in the molar amounts indicated, mixtures of complexes are obtained that comprise, inter alia, the compounds listed below.

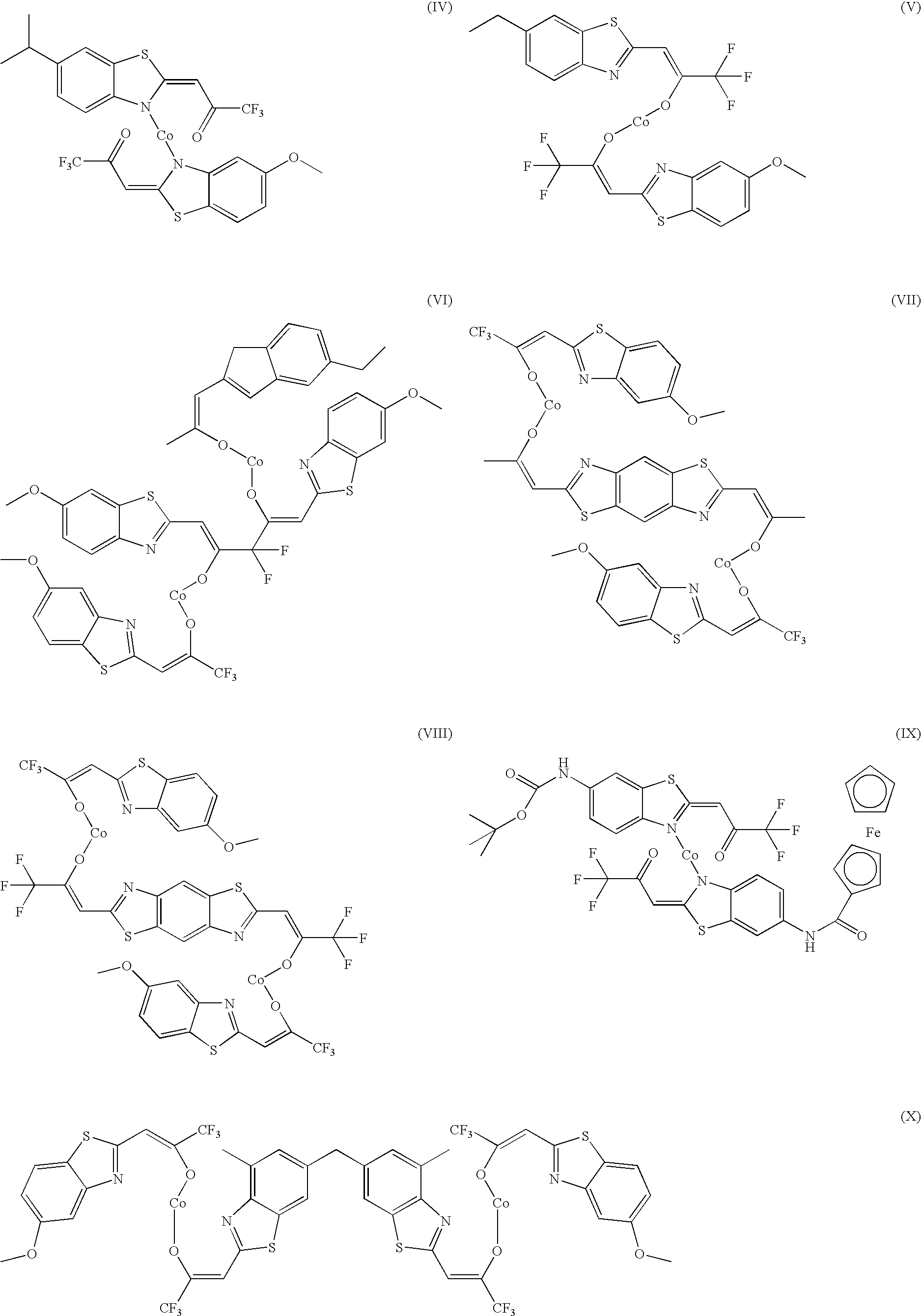

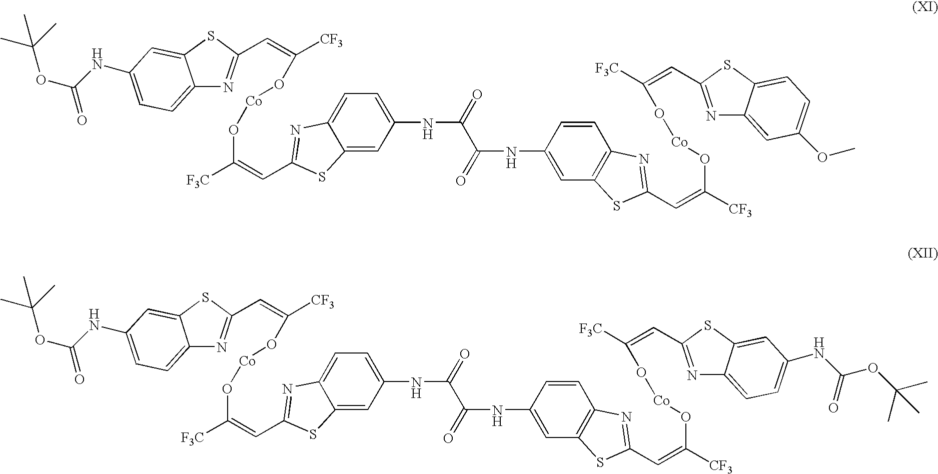

| | Ligands | Molar | | λₘₐₓ | ε | |
|---|---|---|---|---|---|---|
| Ex. | from Ex. | ratio | Structures | [nm] | [l · mol⁻¹ · cm⁻¹] | Solv. |
| 108 | 4 + 6 | 1:1 | Ex. 12 + Ex. 15 + (IV) | 351 | 37200 | CH₂Cl₂ |
| 109 | 4 + 48 | 1:1 | Ex. 12 + Ex. 73 + (V) | 344 | 21440 | CH₂Cl₂ |
| 110 | 4 + 55 | 20:1 | Ex. 12 + (VI) | 356 | 39020 | CH₂Cl₂ |
| 111 | 4 + 55 | 10:1 | Ex. 12 + (VI) | 356 | 39680 | CH₂Cl₂ |
| 112 | 4 + 57 | 20:1 | Ex. 12 + (VII) | 355 | 36020 | CH₂Cl₂ |
| 113 | 4 + 57 | 10:1 | Ex. 12 + (VII) | 354 | 34050 | CH₂Cl₂ |
| 114 | 4 + 56 | 20:1 | Ex. 12 + (VIII) | 355 | 29630 | CH₂Cl₂ |
| 115 | 4 + 56 | 10:1 | Ex. 12 + (VIII) | 355 | 32870 | CH₂Cl₂ |
| 116 | 42 + 51 | 19:1 | Ex. 68 + Ex. 80 + (IX) | 353 | 47200 | CH₂Cl₂ |
| 117 | 42 + 51 | 9:1 | Ex. 68 + Ex. 80 + (IX) | 353 | 47110 | CH₂Cl₂ |
| 118 | 42 + 51 | 4:1 | Ex. 68 + Ex. 80 + (IX) | 354 | 45950 | CH₂Cl₂ |
| 119 | 4 + 58 | 20:1 | Ex. 12 + (X) | 354 | 33860 | CH₂Cl₂ |
| 120 | 4 + 58 | 10:1 | Ex. 12 + (X) | 354 | 38100 | CH₂Cl₂ |
| 121 | 4 + 59 | 20:1 | Ex. 12 + (XI) | 359 | 38500 | DMF |
| 122 | 4 + 59 | 10:1 | Ex. 12 + (XI) | 360 | 38990 | DMF |
| 123 | 4 + 59 | 5:1 | Ex. 12 + (XI) | 362 | 39710 | DMF |
| 124 | 42 + 59 | 20:1 | Ex. 68 + (XII) | 359 | 45280 | DMF |
| 125 | 42 + 59 | 10:1 | Ex. 68 + (XII) | 359 | 45800 | DMF |
| 126 | 42 + 59 | 5:1 | Ex. 68 + (XII) | 361 | 47680 | DMF |

### EXAMPLE 127

Proceeding according to Example 19, but using a 4:1-molar mixture of the compounds according to Examples 4 and 5, there is obtained a mixture of the compounds according to Examples 12, 86 and of the following structure:

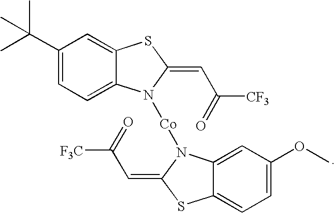

UV/VIS(CH₂Cl₂): λₘₐₓ=353 nm; ε=35 200 l·mol⁻¹·cm⁻¹.

### EXAMPLE 128

200 mg of the compound from Example 12 and 200 mg of the compound from Example 32 are dissolved in 20 ml of CH₂Cl₂; the solution is then concentrated by evaporation and the residue is dried at 40° C./2.5·10³ Pa. A mixture having the following UV/VIS(CH₂Cl₂) is obtained in quantitative yield:

λₘₐₓ=361 nm; ε=41350 l·mol⁻¹·cm⁻¹.

### EXAMPLE 129

0.5 g of the compound from Example 4 and 1.22 g of bis(1,5-cyclooctadienyl)-diiridium(I) dichloride in 20 ml of CH₂Cl₂ are introduced into a 50 ml multi-necked flask equipped with a magnetic stirrer, thermometer, reflux condenser and nitrogen transfer line, and stirring is carried out at reflux for 6 hours under a nitrogen atmosphere. The solution is then concentrated by evaporation and the residue is suspended in 20 ml of water. The suspension is filtered, washed 3× using 10 ml of water each time and dried for 18 hours at 40° C./2.5·10³ Pa. The residue is dissolved in the smallest possible amount of CH₂Cl₂, covered with a layer of hexane and left to stand for 18 hours. The resulting suspension is filtered and washed in portions with 10 ml of hexane and the residue is dried for 18 hours at 40° C./2.5·10³ Pa. A yellowish brown product of the following structure is obtained in a yield of 32%:

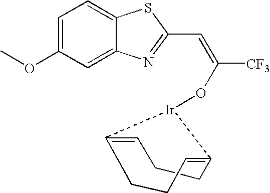

UV/VIS(CH₂Cl₂): λₘₐₓ=375 nm; ε=11 000 l·mol⁻¹·cm⁻¹.

### EXAMPLE 130

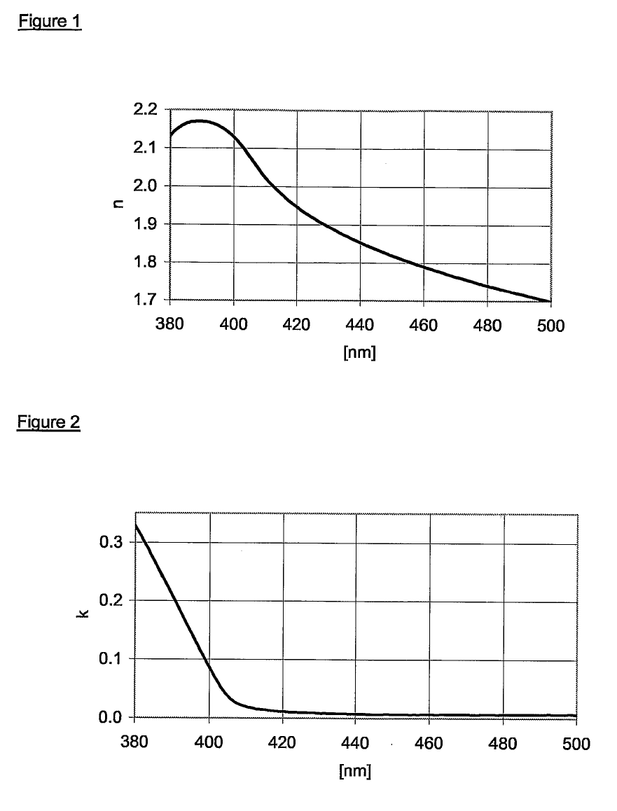

1 g of the compound according to Example 12 is dissolved in 99 g of dichloromethane and filtered through a 0.2 μm Teflon filter. The dye solution is then applied to a 1.2 mm thick, planar glass plate (diameter 120 mm) by spin-coating at 250 rev/min. The excess of the solution is spun off at 2500 rev/min and a uniform solid layer is obtained. After drying, the solid layer has an absorption of 0.26 at 362 nm. Using an optical measuring system (ETA-23° C., STEAG ETA-Optik), a layer thickness of 16 nm and, at 405 nm, a refractive index n of 2.08 and an extinction coefficient k of 0.040, are determined. FIG. 1 shows the refractive index n as a function of the wavelength. FIG. 2 shows the extinction coefficient k as a function of the wavelength.

### EXAMPLE 131

Using a vacuum coating apparatus (Twister™, Balzers Unaxis), a 30 nm thick reflection layer of silver is applied to a 0.6 mm thick grooved polycarbonate disc (diameter 120 mm, groove depth 20 nm, track width 140 nm, track spacing 320 nm). 100 ml of a solution containing 3.0 g of the compound according to Example 12 in 1-methoxy-2-propanol is applied over the reflection layer by spin-coating. After drying (20 minutes, 75° C.), the solid layer has an absorption of 0.80 at 354 nm. A 40 nm thick layer of silicon oxynitride (SiON [12594-30-8]) is applied thereto by means of reactive sputtering (Sprinter™, Unaxis Balzers). To protect the recording layer, a 100 μm thick adhesive polycarbonate film (PC75 HC5LS076KP, Lintec Co./JP) is laminated over the sputtered SiON layer. At 405 nm the recording layer has a high reflectivity. Using a laser disc test apparatus (ODU 1000/Pulstec) of 407 nm wavelength, marks are written into the active layer at a numerical aperture of 0.85, a power of 5 mW and a linear speed of 5.28 m·s⁻¹. The operation brings about a clear reduction in reflection at the written sites (modulation I8/I8H 0.47; 8T CNR: 49 dB; I2 pp/I8 pp: 0.13). The marks are very accurate and readily readable.

It is also possible, for example, for a track width of 120 nm or 160 nm, a power of 7-12 mW or a 50 nm or 100 nm thick reflection layer to be used, an absorption, for example, of 0.34, 0.48 or 0.67 being obtained by variation of the speed of rotation in the spinning-off operation. It is possible to use as dielectric, instead of SiON, for example SiO/SiO₂ or ZnS/SiO₂. It will be understood that other known protecting layers (also directly over the recording layer) are also possible, but preferably as far as possible no chemicals or solvents that are aggressive towards the recording layer will be used and attention will be paid to rapid crosslinking of the compositions in question.

### EXAMPLES 132-135

The compounds according to Examples 17, 19, 68 and 108 are used analogously to Example 131 for the purpose of optical recording. The dynamic test data indicated in Table 1 are obtained:

TABLE 1
Dynamic test data

| | Substance | Optical | Writing | | | |
|---|---|---|---|---|---|---|
| | according | density of | power | | 8T CNR | |
| Example | to Example | the layer | [mW] | l8pp/l8 H | [dB] | l2pp/l8pp |
| 132 | 17 | 0.80 | 7 | 0.42 | 43 | 0.28 |
| 133 | 19 | 0.80 | 5 | 0.41 | 42 | |
| 134 | 68 | 0.74 | 4 | 0.45 | 46 | 0.18 |
| 135 | 108 | 0.80 | 5 | 0.48 | 48 | 0.13 |

Using the media according to the invention, high modulation, high reflection and high sensitivity are simultaneously achieved at lower laser energy.

1. An optical recording medium, comprising a substrate, a recording layer and, optionally, a reflecting layer, wherein the recording layer comprises a compound of formula Mⁿ⁺(L₁)(L₂)_{y}(L₃)_{z} (I) wherein
M is a transition metal of Groups 6 to 12 or an element of Group 13 that may additionally be coordinated with one or more further ligands and/or may optionally have an electrostatic interaction with one or more further ions inside or outside the coordination sphere in order to balance an excess charge;
n is a number 1, 2 or 3; y is the number 0 when n is 1, or is a number 0 or 1 when n is 2 or 3; z is the number 0 when n is 1 or 2, or is a number 0 or 1 when n is 3;
L₁ and L₂ are each independently of the other a ligand of formula 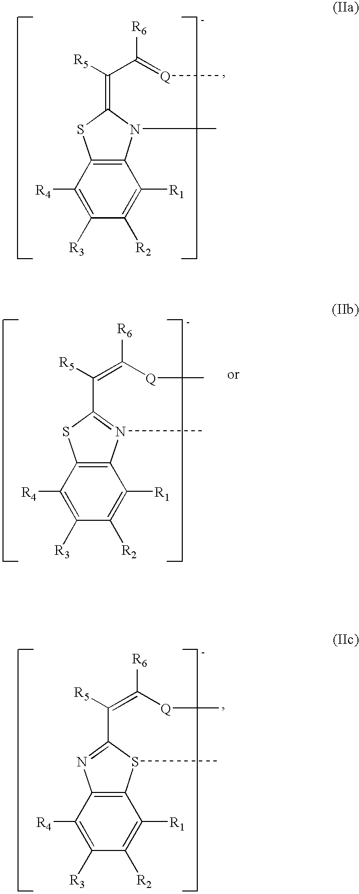
it being possible for L₁ and L₂ to be bonded to one another by any R₁, R₂, R₃, R₄, R₅, R₆ or Q;
L₃, independently of L₁ and L₂, is a further ligand (IIa), (IIb) or (IIc);
Q is O, S, NR₇, N—OR₈ or N—NR₈R₉;
R₁, R₂, R₃ and R₄ are each independently of the others R₁₀, NR₈R₉, NR₁₁NR₈R₉, NO₂, SiR₈R₁₂R₁₃, C(R₁₁)═NR₈, C(R₁₁)═N—OR₈, CON(R₁₁)OR₈, CON(R₁₁)NR₈R₉, S(O)R₁₂, S(O)₂—R₁₂, S(O)—OR₈, S(O)N(R₁₁)NR₈R₉, SO₂NR₈R₉, SO₂N(R₁₁)NR₈R₉, SO₃R₈, P(O)R₁₂R₁₃, P(O)R₁₂OR₈, P(O)OR₈OR₉ or P(O)(NR₈R₉)₂; it being possible for one of R₂, R₃ and R₄ in addition to be C₆-C₁₀aryl, C₁-C₉heteroaryl, C₇-C₁₂aralkyl or C₂-C₁₂heteroaralkyl each unsubstituted or substituted by one or more optionally identical or different nitro, R₁₀ and/or R₇ radicals;
R₅ and R₁₀, independently of R₁ to R₄, and, where applicable, each R₁₀ independently of any other R₁₀, are hydrogen, halogen, OR₇, SR₇, NR₇R₈, COR₁₁, COOR₁₁, CONR₈R₉, CN, OCN or SCN, or C₁-C₅alkyl, C₂-C₅alkenyl, C₂-C₅alkynyl, C₃-C₅cycloalkyl, hetero-C₂-C₅cycloalkyl or C₃-C₅cycloalkenyl each unsubstituted or mono- or poly-substituted by halogen and/or by OR₁₁;
R₆, independently of R₁ to R₅, is hydrogen, OR₈, SR₈, NR₈R₉; C₁-C₅alkyl, C₂-C₅alkenyl, C₂-C₅alkynyl, C₃-C₅cycloalkyl, hetero-C₂-C₅cycloalkyl or C₃-C₅cycloalkenyl each unsubstituted or mono- or poly-substituted by COR₁₁, COOR₁₁, CONR₈R₉, CN, halogen and/or by OR₁₁; or C₆-C₁₀aryl, C₁-C₉heteroaryl, C₇-C₁₂aralkyl or C₂-C₁₂heteroaralkyl each unsubstituted or substituted by one or more optionally identical or different nitro, R₁₀ and/or R₇ radicals;
R₇ is hydrogen, COR₁₁, COOR₁₂, CR₈OR₉OR₁₁, CONR₈R₉, SO₂R₁₂, P(O)R₁₂R₁₃, P(O)R₁₂OR₁₃ or P(O)OR₁₂OR₁₃, or C₁-C₅alkyl, C₂-C₅alkenyl, C₂-C₅alkynyl, C₃-C₅cycloalkyl, hetero-C₂-C₅cycloalkyl or C₃-C₅cycloalkenyl each unsubstituted or mono- or poly-substituted by halogen and/or by OR₁₁;
or R₁ and R₂, R₂ and R₃ and/or R₃ and R₄, in each case together as a pair, are C₂-C₁₀alkylene or C₂-C₁₀alkenylene each unsubstituted or mono- or poly-substituted by halogen and/or by OR₁₁, additional rings being formed that are not fully conjugated, or are 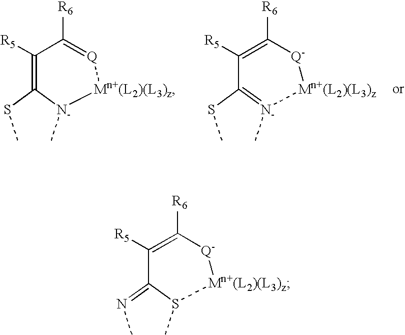
or R₅ and R₆ and/or R₆ and R₇, in each case together as a pair, are C₂-C₁₀alkylene or C₂-C₁₀alkenylene each unsubstituted or mono- or poly-substituted by halogen and/or by OR₁₁, additional rings, but not fully conjugated rings, being formed;
R₈, R₉ and R₁₁, each independently of the others and of R₁ to R₇, are hydrogen; C₁-C₅alkyl, C₂-C₅alkenyl, C₂-C₅alkynyl, C₃-C₅cycloalkyl, hetero-C₂-C₅cycloalkyl or C₃-C₅cycloalkenyl each unsubstituted or mono- or poly-substituted by halogen and/or by OR₁₁; or C₆-C₁₀aryl, C₁-C₉heteroaryl, C₇-C₁₂aralkyl or C₂-C₁₂heteroaralkyl each unsubstituted or substituted by one or more optionally identical or different halogen, OR₁₂, SR₁₂, NR₁₂R₁₃, CN, OCN, SCN, COR₁₂, CR₁₄OR₁₂OR₁₃, COOR₁₂, CONR₁₂R₁₃, SO₂R₁₂, P(O)R₁₂R₁₃, P(O)R₁₂OR₁₃ and/or P(O)OR₁₂OR₁₃ radicals;
or R₇ and R₈ and/or R₈ and R₉ together are C₂-C₁₀alkylene or C₂-C₁₀alkenylene, each unsubstituted or mono- or poly-substituted by halogen and/or by OR₁₁, and each of which may be interrupted by O or by NR₁₁; and
R₁₂, R₁₃ and R₁₄, each independently of the others, are C₁-C₅alkyl, C₂-C₅alkenyl, C₂-C₅alkynyl, C₃-C₅cycloalkyl, hetero-C₂-C₅cycloalkyl or C₃-C₅cycloalkenyl each unsubstituted or mono- or poly-substituted by halogen and/or by OR₁₁, or R₁₂ and R₁₃ together are C₂-C₁₀alkylene or C₂-C₁₀alkenylene each unsubstituted or mono- or poly-substituted by halogen and/or by OR₁₁, and each of which may be interrupted by O or by NR₁₁.

2. An optical recording medium according to claim 1, wherein M is Au, Cd, Co, Cu, Cr, Ir, Mn, Mo, Ni, Fe, Os, Pd, Pt, Re, Rh, Ru, W or Zn.

3. An optical recording medium according to claim 1, wherein n is the number 2, L₁ and L₂ are ligands of formula (IIa) or (IIb) and Q is O or NR₇.

4. An optical recording medium according to claim 1, wherein R₁ is hydrogen or fluorine; R₅ is hydrogen; and/or at least two of R₁, R₂, R₃ and R₄ are hydrogen.

5. An optical recording medium according to claim 1, wherein R₆ is C₁-C₅alkyl, C₂-C₅alkenyl, C₂-C₅alkynyl, C₃-C₅cycloalkyl, hetero-C₂-C₅cycloalkyl or C₃-C₅cycloalkenyl mono- or poly-substituted by halogen and/or by OR₁₁.

6. An optical recording medium according to claim 1, wherein alkyl, alkenyl, alkynyl, cycloalkyl, heterocycloalkyl or cycloalkenyl is methyl, ethyl, n-propyl, isopropyl, vinyl, allyl, propargyl, cyclopropyl, 2-oxacyclopropyl or 2-thiacyclopropyl.

7. A method of recording or playing back data, wherein the data on an optical recording medium according to claim 1 are recorded or played back at a wavelength of from 300 to 500 nm.

8. A compound of formula (I) according to claim 1.

9. A compound of formula 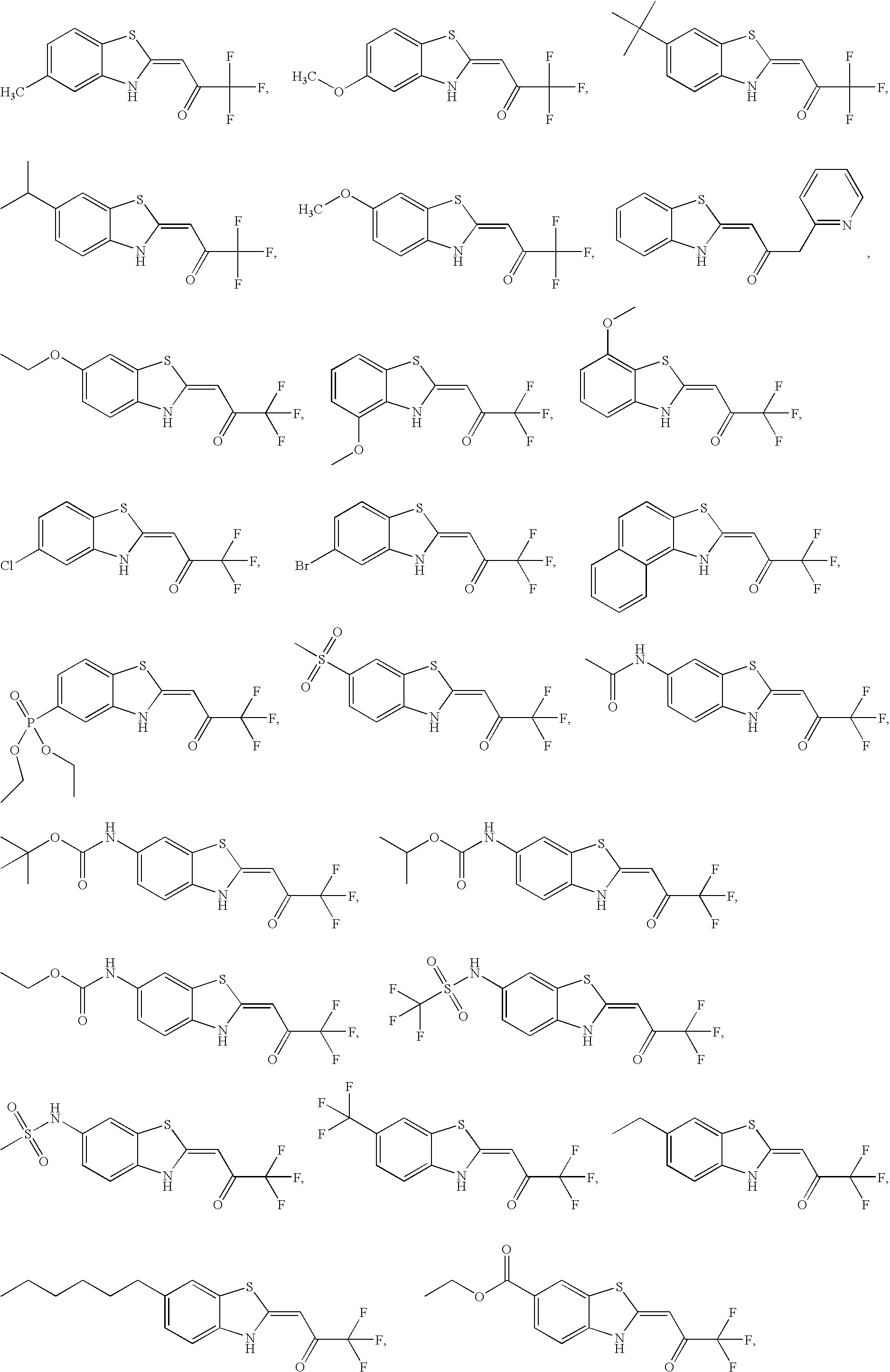 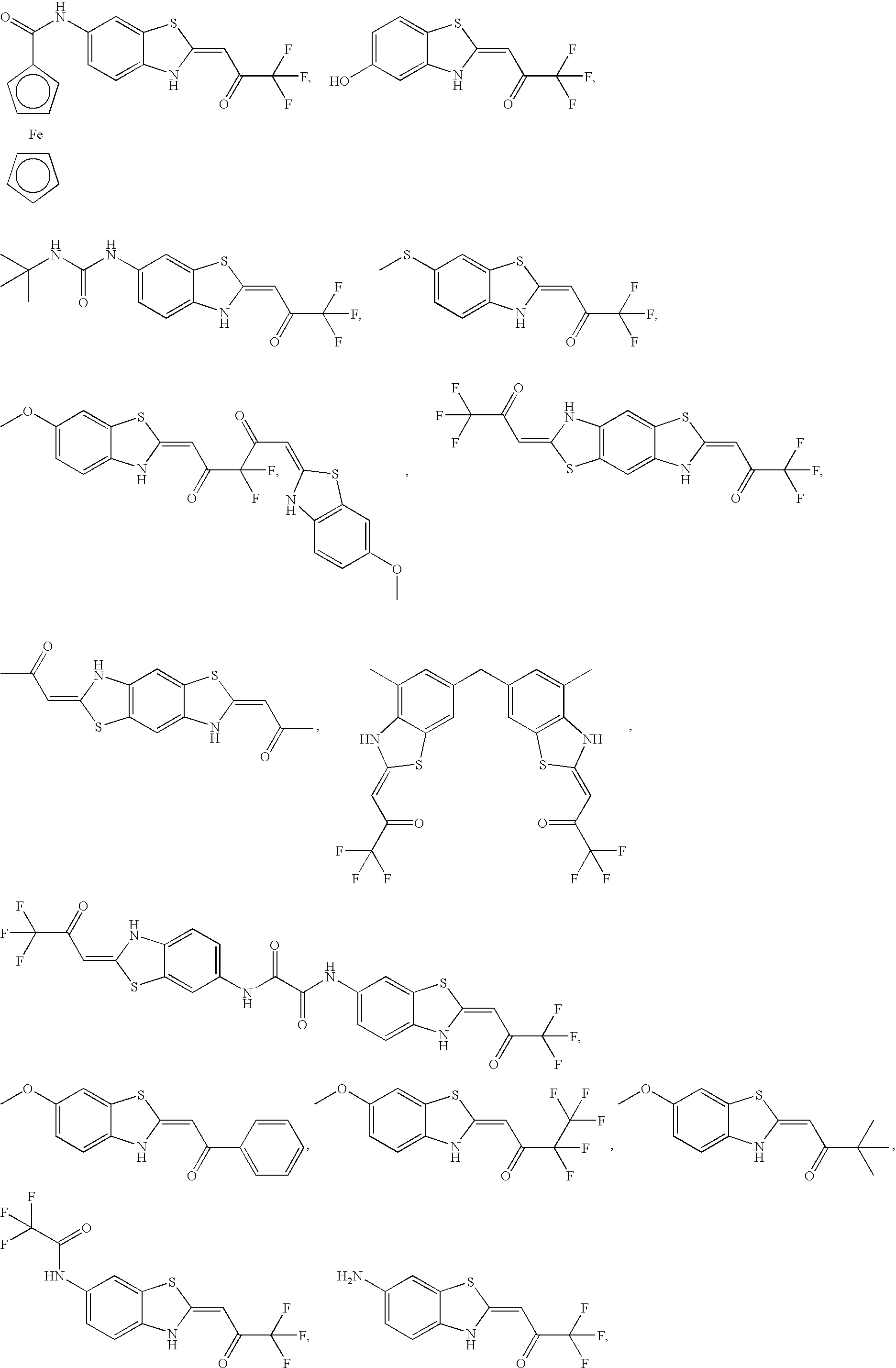 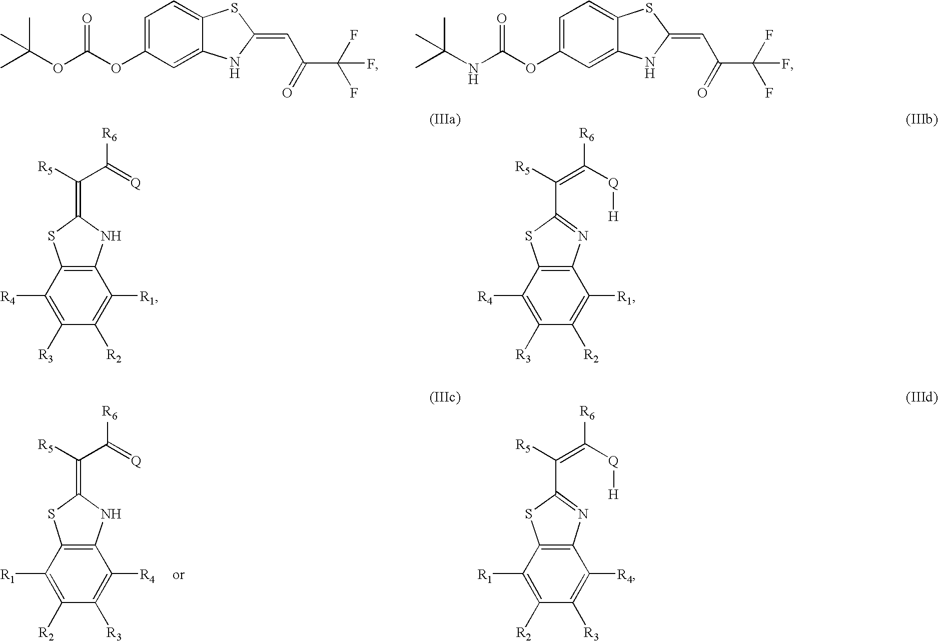
wherein R₆ is a ethyl, n-propyl, isopropyl, C₂-C₅alkenyl, C₂-C₅alkynyl, C₃-C₅cycloalkyl, hetero-C₂-C₅cycloalkyl or C₃-C₅cycloalkenyl radical each of which is unsubstituted or is substituted according to the definitions in formula (I) according to claim 1, with the proviso that there are no H atoms at the C atom by which that radical is bonded to the remainder of formula (IIIa) or (IIIb), and with the exception of the compound wherein R₆ is tert-butyl and R₅ is CN.

10. A method to increase modulation in optical recording media by incorporating a compound according to claim 1 into a recording layer of the optical recording media.

11. An optical recording medium according to claim 2, wherein M is Co, Cu or Ni. more especially Co(II), Cu(II) or Ni(II).

12. An optical recording medium according to claim 11, wherein M is Co(II), Cu(II) or Ni(II).

13. An optical recording medium according to claim 2, wherein n is the number 2, L₁ and L₂ are ligands of formula (IIa) or (IIb) and Q is O or NR₇.

14. An optical recording medium according to claim 2, wherein R₁ is hydrogen or fluorine; R₅ is hydrogen; and/or at least two of R₁, R₂, R₃ and R₄ are hydrogen.

15. An optical recording medium according to claim 2, wherein R₆ is C₁-C₅alkyl, C₂-C₅alkenyl, C₂-C₅alkynyl, C₃-C₅cycloalkyl, hetero-C₂-C₅cycloalkyl or C₃-C₅cycloalkenyl mono- or poly-substituted by halogen and/or by OR₁₁.

16. A compound of formula (I) according to claim 8, wherein M is Au, Cd, Co, Cu, Cr, Ir, Mn, Mo, Ni, Fe, Os, Pd, Pt, Re, Rh, Ru, W or Zn, n is the number 2, L₁ and L₂ are ligands of formula (IIa) or (IIb) and Q is O or NR₇.

17. A compound of formula (I) according to claim 16, wherein R₁ is hydrogen or fluorine; R₅ is hydrogen; and/or at least two of R₁, R₂, R₃ and R₄ are hydrogen.

18. A compound of formula (I) according to claim 17 wherein R₆ is C₁-C₅alkyl, C₂-C₅alkenyl, C₂-C₅alkynyl, C₃-C₅cycloalkyl, hetero-C₂-C₅cycloalkyl or C₃-C₅cycloalkenyl mono- or poly-substituted by halogen and/or by OR₁₁.

19. A method to increase modulation in optical recording media by incorporating a compound according to claim 9 into a recording layer of the optical recording media.
